(19) United States
(12) Patent Application Publication
(10) Pub. No.: US 2022/0339187 A1
(43) Pub. Date: Oct. 27, 2022

(54) ANTIMICROBIAL PEPTIDE-SELENIUM NANOPARTICLES

(71) Applicant: The University of Melbourne, The University of Melbourne, Victoria (AU)

(72) Inventors: Tao Huang, The University of Melbourne (AU); Andrea Janet O'Connor, The University of Melbourne (AU); Neil Martin O'Brien-Simpson, The University of Melbourne (AU); Daniel Edward Heath, The University of Melbourne (AU)

(21) Appl. No.: 17/620,147

(22) PCT Filed: Jun. 19, 2020
(86) PCT No.: PCT/AU2020/050625

§ 371 Date: Dec. 17, 2021

(30) Foreign Application Priority Data
Jun. 21, 2019 (AU) 2019902168

(51) Int. Cl. A61K 33/04; A61K 38/02; A61P 31/04; A61P 31/10; A61P 31/12; C07K 14/36; C07K 17/14

(52) U.S. Cl. CPC A61K 33/04; A61K 38/02; A61P 31/04; A61P 31/10; A61P 31/12; C07K 14/36; C07K 17/14; A61F 13/00063

(57) ABSTRACT

An antimicrobial agent comprising a selenium nanoparticle (SeNP) core and one or more superficially located antimicrobial peptide/s (AMP). The or each AMP may comprise an excess of positively charged amino acids compared to negatively charged amino acids and the AMP may comprise peptides from classes selected from polylysine, such as ε-poly-L-lysine (ε-PL), polyarginine, aurein, ovispirin, melittin, magainin, cecropin, andropin, moricin, ceratotoxin, melittin, magainin, dermaseptin, bombinin, brevinin, esculentins, buforin, cathelicidin, abaecin, apidaecin, prophenin and indolicidin. Products comprising such agents, methods of producing the agents and methods of killing or retarding growth of microorganisms exposed to the agents are also disclosed.

### SUMMARY OF THE INVENTION

The present invention relates to agents that exhibit antimicrobial activity and in particular to agents comprising a selenium nanoparticle (SeNP) core and one or more superficially located antimicrobial peptide/s (AMP). The invention also relates to products comprising such agents, to methods of producing the agents and to methods of killing or retarding growth of microorganisms exposed to the agents.

### BACKGROUND OF THE INVENTION

Cellular (especially microbial) contamination of surfaces is a world-wide problem facing a number of industrial and public health sectors. For example, the attachment of bacterial cells to the inside of metal pipes can lead to biologically induced corrosion and pitting, which may compromise pipe integrity. Attachment of pathogenic bacteria to titanium or other surgical implants can lead to serious post-operative complications, and may result in systemic infections. Food-processing equipment must be maintained free of food-borne pathogens such as Escherichia coli and Salmonella typhimurium to avoid adverse health outcomes. The negative consequences of microbial surface colonization and contamination can be very significant, causing substantial financial losses, severely impacting on human health and, in some cases, resulting in fatality.

The development of agents that are lethal to microbes or exhibit antimicrobial and particularly antibacterial activity, or that reduce growth and/or propagation of microbes, is of commercial importance in a range of contexts. For example, the use of synthetic biocidal surfaces would have great utility and would contribute to efficiencies, improved health outcomes, reduced disease transmission and/or cost savings in areas such as health care provision and surgery, public health, sanitation and domestic hygiene, food processing, preparation, production and storage, animal husbandry, veterinary clinics, aged care facilities, schools and child care facilities, plumbing fixtures, waste treatment and recycling, amongst many others. Specifically, significant advantages can be envisaged in employing antimicrobial surfaces in fittings, devices and apparatus such as walls, floors, ceilings, hand rails, door knobs and handles, seat covers, tables, chairs, light switches, toilets, taps and other surfaces in public, commercial or domestic environments, food preparation surfaces, cooking and food preparation utensils and devices, food and beverage packaging and storage containers, food wrap, medical, surgical and dental tools, instruments and equipment, medical, dental and veterinary implants, hospital surfaces such as floors, walls, sinks, basins, bench tops, beds, mattress and pillow covers, hospital furniture, surgical, medical and food preparation gloves, hair dressing tools and equipment such as combs, brushes, razors and scissors, surfaces in commercial and domestic kitchens such as floors, walls, sinks, basins and bench tops, food and beverage mixers and processing/packaging devices or machines, food and beverage processing lines, abattoirs, protective clothing, goggles and glasses, water and pipes and tanks and fabric, textiles and clothing, especially protective clothing.

Chemical antiseptics and disinfectants are the current modality for the eradication of bacteria and other microorganisms. In the medical context antibiotics have protected countless lives since being discovered at the beginning of the last century. Besides directly curing infection related diseases, antibiotics have enabled the medical profession to undertake more sophisticated treatments with high risk of infection, such as organ transplantation and cancer chemotherapy [1]. However, the abuse of antibiotics has induced the rapid development of antibiotic-resistance, with the result that previously easily treatable diseases may again be deadly.

The emergence of antibiotic resistance is a major global public health issue and challenge faced by healthcare systems [2], which is compounded by the highly adaptable nature of bacteria and their accelerated evolution brought about by over-prescription of antibiotics, their use in food production and by the processes of natural selection. A World Health Organisation report highlighted that the current and foreseeable conventional antibiotic pipeline is insufficient to meet the rise in antibiotic resistance [3]. Hence, new antimicrobial and especially antibacterial agents are urgently needed.

Nanoparticles are providing one option as new generation antibacterial agents and a range of different nanoparticles have been investigated as antimicrobial agents. Antimicrobial nanoparticles include metallic nanoparticles, metallic oxide nanoparticles, inorganic nanoparticles and organic nanoparticles. For example, metallic nanoparticles include silver (Ag) [4-6], gold (Au) [7] and palladium (Pd) [8] nanoparticles; metallic oxide nanoparticles include silver oxide (Ag₂O) [9], magnesium oxide (MgO) [10], calcium oxide (CaO) [11], zinc oxide (ZnO) [12], titanium dioxide (TiO₂) [13], aluminium oxide (Al₂O₃) [14] and copper oxide (CuO) [15] nanoparticles; inorganic nanoparticles include selenium (Se) [16], sulfur [15], tellurium [16], silicon (Si) [17] and silicon dioxide (SiO₂) [18] nanoparticles; organic nanoparticles include chitosan [20], fullerene (C₆₀), and fullerene-derivative [19, 21] nanoparticles. Because these antimicrobial nanoparticles attack microorganisms in multiple ways [22], it is difficult for microorganisms to develop resistance to them.

Up to now silver nanoparticles (Ag NP) are the most extensively studied and used antimicrobial nanoparticles, because they exhibit effective broad-spectrum antibacterial activity. However, toxicity of silver nanoparticles has also been reported [23, 24]. Unlike Ag NPs, selenium is a nutritional element in mammals [25]. In previous work by the present inventors it was demonstrated that at appropriate concentrations, selenium nanoparticles (Se NPs) promote human dermal fibroblast proliferation. Effective antibacterial activity against Gram-positive bacteria like methicillin-sensitive Staphylococcus aureus (MSSA) and methicillin-resistant Staphylococcus aureus (MRSA) has also been found. However, Se NPs have typically shown only very slight antibacterial activity against Gram-negative bacteria. Although involving Se nanowires capped with the peptide crustin, rather than involving nanoparticles, Rekha et al [51] demonstrated only poor antibacterial activity against Gram-negative bacteria.

The magainin antimicrobial peptides are small, positively charged amphipathic molecules, first isolated from the skin of the African clawed frog Xenopus laevis [26] and there have since been a range of other AMPs identified, which are generally positively charged and have variable amino acid composition and length (of from about 5 to about 120 amino acids). For example, the magainins exhibit broad-spectrum antimicrobial activity, which can keep wounds on frog skin free from infection.

AMPs are usually assembled and released as a first line of defence in the organisms that produce them (often being released from skin and mucosa) to fight against pathogenic microorganisms [27]. The major antimicrobial mechanism of AMPs is disrupting the negatively charged bacterial cell membrane by virtue of their positive charge. The AMPs at even very low concentration can induce transient pores in cell membranes by fluctuations [28]. These transient pores allow ion conduction but no passage of large molecules. Stable pores form only when a defined concentration of AMPs bind to the cell membrane, which concentration is referred to as the “threshold point” [29]. The extent of disruption of the bacterial cell membrane increases with the peptide concentration [29]. However, high concentrations of AMPs exhibit high toxicity to mammalian cells [30]. Therefore, limited successful clinical applications of AMPs have so far been found.

ε-poly-_{L}-lysine (ε-PL) is a simple natural antimicrobial peptide with 25-30 _{L}-lysine residues [32], which was accidently found and isolated form Streptomyces albulus strain 346 [33]. Although mutant strains of Streptomyces albulus were developed to improve ε-PL production yields, no other bacterial strains or eukaryotes with an ability to produce ε-PL have been identified up to now [33]. ε-PL is being widely used as a food additive, in view of the broad spectrum antimicrobial activity it exhibits against both Gram-positive and Gram-negative bacteria, as well as its anti-fungal activity [34, 35]. Desirably, ε-PL is water soluble and exhibits low toxicity. Hiraki et al. researched acute oral toxicity of ε-PL in mice and found no mortality with a high dosage of 5 g/kg [36]. ε-PL generally exhibits better antibacterial activity against Gram-negative than Gram-positive bacteria [37, 38].

It is with the background discussed above in mind that the inventors have conceived the present invention. Further details, advantages and applications of the invention will become apparent from the following detailed description thereof.

### SUMMARY OF THE INVENTION

In one embodiment of the present invention there is provided an antimicrobial agent comprising a selenium nanoparticle (SeNP) core and one or more superficially located antimicrobial peptide/s (AMP).

In another embodiment of the present invention there is provided an antimicrobial composition comprising the antimicrobial agent referred to above and one or more carrier, diluent or vehicle.

In a further embodiment of the invention there is provided a method of synthesis of an antimicrobial agent that comprises a selenium nanoparticle (SeNP) core and one or more superficially located antimicrobial peptide/s (AMP), said method comprising dispersing Se NPs into a solution of the one of more AMP and recovering the antimicrobial agent produced.

In a still further embodiment of the present invention there is provided a method of killing or retarding growth of a microorganism, comprising exposing a microorganism or its locus to the antimicrobial agent referred to above.

In a further embodiment of the invention there is provided use of the antimicrobial agent as referred to above in preparation of a composition or product that is effective for killing or retarding growth of a microorganism.

In a still further embodiment of the present invention there is provided an article that incorporates the antimicrobial agent referred to above on a surface thereof that may be exposed to a microorganism, or that enables agent release/exposure to a microorganism, wherein said article comprises a cleaning or disinfecting formulation, textile, clothing, furniture, a building fitting or fixture, a food preparation surface, utensil or apparatus, a food or beverage packaging, processing or storage container, food wrap, a wound dressing or a medical, surgical, veterinary or dental implant, tool or instrument.

### BRIEF DESCRIPTION OF THE FIGURES

The invention will be further described with reference to the following figures, wherein:

FIG. 1 (a) provides TEM images of Se NP-ε-PL; FIG. 1 (b) is a size distribution (nm) diagram for Se NP-ε-PL; and FIG. 1 (c) and FIG. 1 (d) show representative zeta potential (mV) distributions for Se NPs and Se NP-ε-PL, respectively.

FIG. 2 shows bar graphs of viability percentage for human dermal fibroblasts exposed to different concentrations of Se NPs (a), Se NP-ε-PL (b) and pure ε-PL (c). A one-way ANOVA followed by Tukey's Post Hoc test was used to compare means of experimental groups to that of the negative control group, * p-value<0.05, ** p-value<0.01 and *** p-value<0.001. The asterisk(s) directly marked on a bar indicate(s) this group is significantly different to all other groups.

FIG. 3 shows bar graphs of LDH release (as percentage of total) for human dermal fibroblasts exposed to different concentrations of Se NPs (a), Se NP-ε-PL (b) and pure ε-PL (c).

FIG. 4 shows growth curves (absorbance (a.u.) against time (hours)) for various bacterial cells in MHB exposed to Se NPs, Se NP-ε-PL or pure ε-PL at a concentration of 12.5 μg/mL, where the bacteria are Staphylococcus aureus (a), MRSA (b), Enterococcus faecalis (c), Escherichia coli (d), A. baumannii (e), Pseudomonas aeruginosa (f), Klebsiella pneumoniae (g) and Klebsiella pneumoniae (MDR) (h).

FIG. 5 shows colony forming unit (CFU) assay results of CFU (mL⁻¹) against concentration (μg/ml) for different bacteria in MHB with different concentrations of Se NPs, Se NP-ε-PL or pure ε-PL: where the bacteria are (a) Staphylococcus aureus, (b) MRSA, (c) Enterococcus faecalis, (d) Escherichia coli, (e) Acinetobacter baumanii, (f) Pseudomonas aeruginosa, (g) Klebsiella pneumoniae and (h) Klebsiella pneumoniae (MDR).One-way ANOVA analysis was adopted to compare means of experimental groups, * represents the P-value<0.05. The asterisk(s) directly marked on a bar indicate(s) this group is significantly different to all other groups at the same concentrations.

FIG. 6 shows bar graphs of ATP concentration (nM) for various bacteria treated with specified concentrations (μg/mL) of Se NPs, Se NP-ε-PL or pure ε-PL, where the bacteria are (a) S. aureus, (b) E. faecalis, (c) E. coli and (d) K. pneumoniae, with bacteria in pure MHB as a control. One-way ANOVA analysis was adopted to compare means of experimental groups, * represents the P-value<0.05, ** represents the P-value<0.01 and *** represents the P-value<0.001. The asterisk(s) directly marked on a bar indicate(s) this group is significantly different to all other groups at the same concentrations.

FIG. 7 shows bar graphs of percentage of high Reactive Oxygen Species (ROS) production cells in (a) S. aureus, (b) E. faecalis and (c) E. coli treated with Se NPs, Se NP-ε-PL or pure ε-PL at 6.25 μg/ml or 12.5 μg/ml, with bacteria in pure MHB as a control. One-way ANOVA analysis was adopted to compare means of experimental groups, * represents the P-value<0.05, ** represents the P-value<0.01 and *** represents the P-value<0.001. The asterisk(s) directly marked on a bar indicate(s) this group is significantly different to all other groups at the same concentrations.

FIG. 8 shows bar graphs of the percentage of depolarized (a) S. aureus, (b) E. faecalis and (c) E. coli cells after treatment with Se NPs, Se NP-ε-PL or pure ε-PL at 6.25 μg/ml or 12.5 μg/ml. A one-way ANOVA analysis followed by Tukey's Post Hoc test was adopted to compare means of experimental groups, * represents the P-value<0.05, ** represents the P-value<0.01 and *** represents the P-value<0.001. The asterisk(s) directly marked on a bar indicate(s) this group is significantly different to all other groups at the same concentrations.

FIG. 9 shows bar graphs of the percentage of propidium iodide (PI) positive bacteria cells after treatment with Se NPs, Se NP-ε-PL or pure ε-PL at 6.25 μg/ml or 12.5 μg/ml, where the cells are S. Aureus (a), E. faecalis (b), E. coli (c), A. baumannii (d), P. aeruginosa (e) and K. pneumonia (f). One-way ANOVA analysis was adopted to compare means of experimental groups, * represents the P-value<0.05, ** represents the P-value<0.01 and *** represents the P-value<0.001. The asterisk(s) directly marked on a bar indicate(s) this group is significantly different to all other groups at the same concentrations.

FIG. 10 shows helium ion microscopy images of S. aureus, E. faecalis, E. coli, A. baumannii and K. pneumoniae with Se NPs, Se NP-ε-PL or pure ε-PL, with bacteria in pure MHB as a control. Pink arrows indicate Se NP-ε-PL attached to the bacteria.

FIG. 11 provides resistance development plots (fold change in MBC against bacterial growth generation) for (a) Se NPs, Se NP-ε-PL and kanamycin on S. aureus; and (b) Se NP-ε-PL and kanamycin on E. coli.

FIG. 12 shows a schematic of the hypothesized mechanism of antibacterial action of Se NP-ε-PL. The Se NP-ε-PL can easily attach to the bacterial cell membrane through electrostatic interactions. The Se NP-ε-PL will then damage the bacterial cell through promoting reactive oxygen species (ROS) production, depleting ATP, changing membrane potential and disrupting the membrane. The Se NP-ε-PL has the potential to induce DNA damage and protein damage directly or as a result of the high levels of ROS.

### DETAILED DESCRIPTION OF THE INVENTION

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Documents referred to within this specification are included herein in their entirety by way of reference.

The reference to any prior art in this specification is not, and should not be taken as, an acknowledgment or any form of suggestion that that prior art forms part of the common general knowledge in Australia.

The present inventors have now demonstrated that not only does an antimicrobial agent comprising a selenium nanoparticle (SeNP) core and one or more superficially located antimicrobial peptide/s (AMP) exhibit broad spectrum antibacterial activity against both Gram-positive and Gram-negative bacteria and other microbes, it exhibits activity against antibiotic resistant strains and is far less susceptible to the development of bacterial resistance than existing antibacterial agents. It is also surprising, and could not have been predicted by a skilled person, that when attached superficially to a SeNP core, both the attached AMP and the SeNP are able to exhibit inter-related or coordinated antimicrobial activity.

The core or central component of the antimicrobial agents of the present invention are selenium nanoparticles, referred to throughout this specification as SeNP. SeNP are well characterized and readily produced, for example by reduction of readily commercially available selenite precursor compounds such as SeO₂, SeO₃²⁻, H₂SeO₃, Ag₂SeO₃ or Na₂SeO₃ by conventional reducing agents such as sodium borohydride, hydrogen peroxide, iron sulfate, sodium dithionate, sodium thiosulfate, ascorbic acid, glutathione and the like. SeNP can readily be produced by reduction of selenium dioxide with sodium thiosulfate as further discussed in the example below.

The morphology, shape and size distribution of SeNP can be varied in a controlled manner depending upon the route of production adopted for synthesis. For example, nanometer-sized particles of amorphous selenium can be produced by exposure of selenious acid to gamma-radiation, by electrochemical oxidation of the selenide ion and by reduction of selenious acid. In an example of the latter approach a stable dispersion of uniform and amorphous selenium particles with a size of about 100 nm can be produced by the reduction of selenious acid solution with hydrazine hydrate in the presence of poly(vinylpyrrolidone) (PVP) [31]. Further addition of a solvent with low polarity such as n-butyl alcohol into this aqueous solution and mild stirring results in the transportation of amorphous selenium particles onto a liquid-liquid interface between water and n-butyl alcohol. The subsequent crystallization and shape evolution on this interface results in the formation of crystalline selenium nanorods [31].

In other approaches, SeNP can be produced by reduction of sodium selenite with ascorbic acid, with stabilization with chitosan to produce spherical nanoparticles of about 200 nm diameter or stabilization by folic acid-gallic acid-N,N,N-trimethylammonium chitosan (FA-GA-TMC) to produce cube-like structured SeNPs of about 300 nm in size [52].

Although they may take a variety of forms such as needle-like, cube-like, fiber-like, rod-like or spherical, in one aspect of the invention the SeNPs are substantially spherical. This is a suitable shape for the purposes of production of the present antimicrobial agents as in this form superficially located AMP are readily presented to adjacent microbial cell surfaces. Nanowires, which may be sized in the nano scale in two dimensions, but which have an extensive third dimension (length) are generally not considered to constitute nanoparticles.

Another key component of the agents of the invention is one or more antimicrobial peptide (AMP), which are generally, although not necessarily, positively charged and have variable amino acid composition and length (of from about 5 to about 120 amino acids). In the context of the present invention the term AMP is intended to encompass any peptide formed from naturally occurring and/or non-proteinogenic or non-naturally occurring amino acids, which exhibits anti-microbial, and particularly anti-bacterial activity. Those AMPs that are positively charged will inherently include an excess of positively charged amino acids in comparison to negatively charged amino acids. In the case of positively charged AMPs comprising naturally-occurring amino acids there will be an excess of positively charged amino acids selected from arginine, lysine and histidine in comparison to negatively charged amino acids selected from aspartic acid and glutamic acid. In one aspect of the invention the combined SeNPs and AMP molecules have an overall net positive charge, which without wishing to be bound by theory, the present inventors consider likely to contribute to the efficacy of agents of the invention exhibiting strong antibacterial activity against Gram-negative bacterial as well as against Gram-positive bacteria.

Examples of suitable AMP classes include, but are not limited to polylysine, polyarginine, aurein, ovispirin, melittin, magainin, cecropin, andropin, moricin, ceratotoxin, melittin, magainin, dermaseptin, bombinin, brevinin, esculentins, buforin, cathelicidin, abaecin, apidaecin, prophenin and indolicidin. In one aspect of the invention the AMP comprises ε-poly-_{L}-lysine (ε-PL).

The agents of the present invention comprise a SeNP core and one or more superficially located AMP molecules, By the term “superficially” it is intended to denote that the AMP molecules are accessible at the surface of the agent to interact with microbes. The AMP molecule/s can be attached to the SeNP core by a variety of means herein further discussed but in each case, in order to demonstrate their antimicrobial character, the AMP molecules must be available at the surface of the SeNP core to interact with microbes, such as by disrupting the membrane of bacterial or other microbial cells. The SeNP cores in a sample of the antimicrobial agent will include at least one AMP molecule and will preferably include multiple AMP molecules. The AMP molecules can be the same or different, such that in a sample of the antimicrobial agent of the invention there can be a single type of AMP, two or more types of AMP on each SeNP core or collections of SeNP cores having single or multiple types of AMP with other SeNP cores having another or other types of SeNP cores.

For example, the agents of the invention comprising SeNP core and one or more AMP may have mean particle size (diameter in the case of spherical agents) of from about 10 nm to about 400 nm, about 20 nm to about 350 nm, about 30 nm to about 300 nm, about 40 nm to about 250 nm, about 50 nm to about 200 nm, about 60 nm to about 150 nm, about 70 nm to about 120 nm, about 75 nm to about 100 nm or about 80 nm, 85 nm, 90 nm or 95 nm.

The one or more superficially located AMP molecule/s may be directly attached to the SeNP core by a variety of suitable means or may, for example, be attached through the agency of a stabilizer or linker material. In one aspect of the invention, in the case of generally negatively charged SeNPs as core material and generally positively charged AMP molecules, the AMP molecules can be directly attached to the SeNP surface by electrostatic adsorption. The invention also comprehends the use of conventional stabilizer and/or linker materials that may be employed to be applied to the SeNP surface such that the surface is rendered more amenable for attachment of AMP molecule/s. For example, stabilizers or linkers may be hydrogen rich, ionic or polymeric materials capable of binding to the SeNP core that provide suitable chemistry for ionic, hydrogen or covalent binding of the AMP molecule/s to the SeNP core. AMP molecule/s may be linked to these stabilizers or linkers using, for example, click chemistry, photochemical reactions, or carbodiimide reactions, including reactions of EDC [1-ethyl-3-(3-dimethylaminopropyl) carbodiimide hydrochloride] and sulfo-NHS (N-hydroxy sulfosuccinimide). For example, a stabilizer or linker can be formed from a single material or may be formed through a graded deposition or co-deposition process, for example, wherein there is no defined boundary between SeNP substrate and surface but where there is a gradual change in character from being more Se like to being a surface more compatible for binding to the AMP molecule/s. For example, such graded interfaces between the Se core and the AMP can be generated using a plasma deposition production approach where the plasma generating gas content is progressively changed from being more like the core to a gas that deposits a layer that is more compatible for binding to the AMP.

The agents according to the invention that are lethal to microorganisms and particularly exhibit antibacterial activity, are generally referred to throughout this specification as exhibiting “antimicrobial” activity. Similarly, the agents of the invention can be incorporated into articles, such that they are located within the article at a surface or on surfaces that may come into contact with microbes and/or are released from articles to come into contact with microbes (for example released into surrounding fluid). In this way in use of the article the antimicrobial agents incorporated therein can exhibit their antimicrobial action against microbes that come into contact with the surfaces of the article or with agent released from the article. That is, upon contact to the antimicrobial agent or the surfaces of the articles incorporating them microbes will be killed or at least have their growth retarded. It will be appreciated, however, that although “antimicrobial” the agents of the invention and surfaces of articles incorporating them will not immediately kill all microbes exposed to them. Rather, a period of exposure will be required that will enable a proportion of cells in a microbial cell population exposed to the surface or agent released form the article to physically come into contact with the agent. Therefore, depending upon the concentration of cells exposed to the agent, the duration of exposure and the concentration of agent or surface area of the surface to which they are exposed, the agent may not be lethal to all cells. However, while the agents will be lethal to at least some of the cells, from a cell population perspective reduction in cell growth and/or propagation may be observed. Routine assays to determine cell colony numbers and/or propagation (such as standard plate counts) and staining to identify cell lysis are available to demonstrate antimicrobial activity. Microscopic techniques such as confocal laser scanning microscopy and scanning electron microscopy can also be used to observe the antimicrobial effect of agents according to the invention.

In addition to antimicrobial agents and articles incorporating them the present invention relates to methods of eliminating microbes, reducing microbial survival and/or reducing microbe growth and/or propagation that involve exposing microbes or their locus to the agents, including exposing microbes to agents incorporated in an article or released from an article. Preferably the exposure will be such that a high proportion of any population of microbes intended to be eliminated will have physical access to the agent or surface of the article incorporating it to allow direct interaction between the microbes and the agent. This dynamic can of course be varied by modification of the concentration of agent exposed to the microbe, by modifying the amount per surface area of article to which microbes are exposed, by modification of rate of release of agent from an article and by modification of the duration of exposure. Reduction of microbe growth, propagation and/or survival can readily be determined using techniques referred to above, in comparison to equivalent populations of cells exposed to a control agent. The agents, articles and methods of the invention are effective to eliminate cells, reduce cellular survival, reduce cell growth and/or propagation of a wide variety of cells including both prokaryotic and eukaryotic cells, and specifically Gram-positive and Gram-negative bacteria cells (including spores), fungi cells (including yeasts), protist cells, helminth cells and cells of other microorganisms such as protozoa, archaea, rotifers and planarians. Virus infected cells can also be eliminated according to the invention. The present invention will particularly be adopted for elimination of cells or organisms that are unsightly (such as mold, fungi), malodorous, corrosive, may form biofilms and especially that are a threat to human or animal health, and especially for the elimination or growth retardation of pathogenic Gram-positive or Gram-negative bacteria. Specific examples of pathogenic and non-pathogenic cells or organisms that can be eliminated according to the present invention include, but are not limited to Pseudomonas aeruginosa, Pseudomonas fluorescens Escherichia coli, Branhamella catarrhalis, Planococcus maritimus, Staphylococcus aureus, Bacillus subtilis, Staphylococcus aureus, Staphylococcus aureus (Multidrug resistant — (MDR)), Enterococcus faecalis, Acinetobacter baumanii, Klebsiella pneumoniae and Klebsiella pneumoniae (MDR), Mycobacterium tuberculosis, Neisseria gonorrhoeae, Streptococcus pneumonia and Staphylococcus epidermidis.

Accordingly, in one embodiment, the present invention provides a method of killing or retarding growth of a microorganism, comprising exposing a microorganism or its locus to the antimicrobial agent of the invention. In this context the application of the antimicrobial agent of the invention may involve the treatment of a subject (in which case the locus is the subject or a specific site or intended organ or wound of the subject). The agent may be applied topically to the subject, such as to a wound, or administered to a subject orally or parenterally either to treat an existing microbial infection or to prevent or minimize the pathogenic effect of potential infection. Both treatment and prophylaxis are referred to herein as “treatment” depending upon the context. For example, the agent of the invention may be administered to a subject suffering from a wound, undergoing or following on from surgery, diagnosed with or suspected of having a microbial infection, such as a bacterial infection, where the subject has been or will be exposed to a microbial pathogen or is immunocompromised (e.g. a subject undergoing treatment for another disease or disorder, a subject undergoing cancer chemotherapy, therapy with immunosuppressive agents or who is otherwise prone to microbial infection, such as the old, young or otherwise infirm).

As used herein, the term “subject” refers to an animal, such as a bird or a mammal. Specific animals include rat, mouse, dog, rabbit, guinea pig, cat, cow, sheep, horse, pig or primate. A subject may further be a human, alternatively referred to as a patient.

While the locus of a microorganism constitutes a subject in the context of a medical or veterinary therapy, the agents of the invention may also be applied to other articles, surfaces or materials where it is desired to eliminate or reduce microbial, such as bacterial, contamination. A range of potential articles to which the agents of the invention may be incorporated or applied are recited elsewhere in this document. For application of the agents of the invention in non-medical or non-veterinary treatment contexts the agents can be provided in formulations with conventional carriers or diluents used in disinfecting or antimicrobial formulations, such as water, ethanol and other active ingredients. For example the agents may be incorporated into conventional dishwashing, detergent, floor and surface cleaning, textile washing, hand and body sanitizing, disinfecting, dental care or other cleaning/sterilizing formulations and may be applied to a surface or article by spraying, brushing, wiping etc. as is conventional in cleaning and disinfecting operations. The agents may also be incorporated into conventional coating formulations such as paints, stains, dyes, sealants, anti-corrosive coatings, water-proofing coatings or the like for application to other articles. One such example is a coating formulation to be applied to implantable medical, veterinary or dental devices.

The present invention encompasses pharmaceutical and cosmetic compositions comprising the agent according to the invention, together with at least one pharmaceutically or cosmetically acceptable carrier or diluent. Such compositions or medicaments can readily be prepared by routine cosmetic, pharmaceutical or veterinary methods by bringing the agent into intimate admixture with the carrier and/or diluent.

As will be readily appreciated by those skilled in the art, the route of administration and the nature of the pharmaceutically acceptable carrier will depend on the nature of the condition and the animal (including a human) to be treated. The agents may for example be administered to a subject topically, mucosally, intravenously, enterally (such as orally) or parenterally, as therapeutic and/or prophylactic agents. The choice of a particular carrier or delivery system, and route of administration can readily be determined by a person skilled in the art taking into account the subject's condition, age, weight, gender and general state of health. In the preparation of any formulation containing the agent according to the invention care should be taken to ensure that the activity of the agent is not destroyed in the process and that is able to reach its site of action without in an active form. Similarly, the route of administration chosen should be such that the agent reaches its site of intended action.

Those skilled in the art can readily determine appropriate formulations for the agents of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

It is envisaged that the agents according to the invention will be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems suitable for the agent, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the agents of the invention in the required amount in the appropriate solvent with various of the other ingredients such as those enumerated above, as required, followed by filter sterilization. Generally, dispersions are prepared by incorporating the sterilised agent into a sterile vehicle which contains the basic dispersion medium and the required other ingredients from those enumerated above.

Pharmaceutically acceptable vehicles and/or diluents include any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the agents of the invention, use thereof in the therapeutic compositions is contemplated. Supplementary active ingredients can also be incorporated into the compositions.

It is especially advantageous to formulate the compositions in unit dosage form for ease of administration and uniformity of dosage. Unit dosage form as used herein refers to physically discrete units suited as unitary dosages for the subjects to be treated; each unit containing a predetermined quantity of the agent calculated to produce the desired efficacy in association with the required pharmaceutically acceptable vehicle. The specification for the novel unit dosage forms of the invention are dictated by and directly dependent on (a) the unique characteristics of the agent and the particular efficacy to be achieved, and (b) the limitations inherent in the art of compounding the agent of the invention in living subjects having a diseased condition in which bodily health is impaired.

As mentioned above the agent of the invention may be compounded for convenient and effective administration in effective amounts with a suitable pharmaceutically acceptable vehicle in unit dosage form. A unit dosage form can, for example, contain the nanoparticles in amounts ranging from 0.25 μg to about 2000 μg. Expressed in proportions, the agent may be present in from about 0.25 μg to about 2000 μg/mL of carrier. In the case of compositions containing supplementary active ingredients, the dosages are determined by reference to the usual dose and manner of administration of such ingredients.

As further outlined in the example below, the present invention also encompasses a method of synthesis of an antimicrobial agent that comprises a selenium nanoparticle (SeNP) core and one or more superficially located antimicrobial peptide/s (AMP). In one aspect this method comprises dispersing Se NPs into a solution of the one of more AMP and recovering the antimicrobial agent produced. However, the method adopted will depend upon the means of attaching the AMP to the SeNP core and the specific AMP/s involved. For example, the method may involve depositing a polymer or other linking or stabilizing material onto the surface of the SeNPs and then effecting appropriate chemistry to bind the AMP/s to the core. It will be generally understood that the agents of the invention may be in some way attached or adhered to a substrate article or material or may be integral with the substrate article or material such as by being integrally formed from a single material or by being formed through a graded deposition or co-deposition process, for example, wherein there is no defined boundary between substrate and surface but where there is a gradual change in character from being more substrate material like to being a surface more compatible for binding to the antimicrobial agent. For example, such graded interfaces between a substrate material and the agent can be generated using a plasma deposition production approach where the plasma generating gas content is progressively changed from being more like the substrate material to a gas that deposits a layer that is more compatible for binding to the agent. For example, the agent may be directly bound to the surface of the substrate material (which may be appropriate in the case of polymer substrate materials) or may be affixed to a substrate material by known means such as use of conventional adhesives, heat bonding or the like. Alternatively, a linker or stabilizer agent may be deposited on the surface of the substrate material (such as in the case of metal or ceramic substrates that are less amenable for direct chemical binding).

The agent of the invention may also be incorporated into a coating, sheath or covering that is shaped and sized to readily fit to the substrate article, for example allowing for removable fitting. The agents may also be incorporated into a material, such as a hydrogel, that is degradable and releases agent as it degrades.

Articles/substrates into which agents of the present invention can be incorporated can be formed from a variety of materials such as metal, semiconductor, polymer, composite and/or ceramic materials. Such materials may take the form of a block, sheet, film, foil, tube, strand, fiber, piece or particle (e.g. a nano- or micro-particle such as a nano- or micro-sphere), powder, shaped article, porous article, indented, textured or molded article or woven fabric or massed fiber pressed into a sheet (for example like paper) of metal, semiconductor, polymer, hydrogel, composite and/or ceramic. Depending upon the nature of the material being used to form the nano structured surface the manufacturing process will necessarily be modified. Such materials can form, or can form parts or components of, other devices, tools, fittings or apparatus on the surface of which is it desired to eliminate or at least slow the growth or progression of microbes, and in particular microbes that are infective, pathogenic, malodorous and/or unsightly, for example. Devices, tools, fittings and apparatus according to the present invention include but are not limited to walls, floors, ceilings, hand rails, door knobs, handles, seat covers, tables, chairs, light switches, toilets, taps, sinks, basins, bench tops, beds, mattress and pillow covers, hospital furniture, food preparation surfaces, cooking and food preparation utensils and devices, food and beverage packaging and storage vessels, food wrap, medical, surgical, veterinary and dental tools, instruments and equipment, medical, dental and veterinary implants, gloves, combs, brushes, razors, scissors, food and beverage mixers and processing/packaging devices or machines, food and beverage processing lines, protective clothing, protective masks, goggles and glasses, water and sewerage pipes, tanks and drains, wall and floor tiles and laminates for floors, furniture, walls, bench tops and other surfaces. It is also possible for the agents of the invention to be included directly or applied or incorporated into articles or materials in the form, for example of strands, fibres, pieces or particles for inclusion in polymer production blends and in coating or printing compositions, such as paints, dyes or inks (including 3D printing inks) to form a substrate, or that can be applied to a substrate, to impart antimicrobial activity on the substrate so treated. Fibres, yarns or strands of material to which the agents of the invention have been incorporated can be incorporated in woven fabrics and materials that can in turn be incorporated into article or products such as clothing including protective clothing, drapery, bed linen, furniture coverings, cloths, towels, wound dressings, face masks, bandages and wipes to impart antimicrobial/disinfecting character upon the article or product.

The terms “metal” or “metallic” as used herein to refer to elements, alloys or mixtures which exhibit or which exhibit at least in part metallic bonding. Preferred metals according to the invention include elemental iron, copper, zinc, lead, aluminium, titanium, gold, platinum, silver, cobalt, chromium, vanadium, tantalum, nickel, magnesium, manganese, molybdenum, tungsten and alloys and mixtures thereof. Particularly preferred metal alloys according to the invention include cobalt chrome, nickel titanium, titanium vanadium aluminium and stainless steel.

The term “ceramic” as it is used herein is intended to encompass materials having a crystalline or at least partially crystalline structure formed essentially from inorganic and non-metallic compounds. They are generally formed from a molten mass that solidifies on cooling or are formed and either simultaneously or subsequently matured (sintered) by heating. Clay, glass, cement and porcelain products all fall within the category of ceramics and classes of ceramics include, for example, oxides, silicates, silicides, nitrides, carbides and phosphates. Particularly preferred ceramic compounds include magnesium oxide, aluminium oxide, hydroxyapatite, titanium nitride, titanium carbide, aluminium nitride, silicon oxide, zinc oxide and indium tin oxide.

The term “semiconductor” as it is used herein refers to materials having higher resistivity than a conductor but lower resistivity than a resistor; that is, they demonstrate a band gap that can be usefully exploited in electrical and electronic applications such as in diodes, transistors, and integrated circuits. Examples of semiconductor materials include silicon, silicon dioxide (silica), germanium, gallium arsenide, indium antimonide, diamond, amorphous carbon and amorphous silicon. If silicon is utilised the silicon may be doped silicon, such as boron doped silicon.

“Composite” materials comprehended by the present invention include those that are combinations or mixtures of other materials, such as composite metallic/ceramic materials (referred to as “cermets”) and composites of polymeric material including some metallic, ceramic or semiconductor content, components or elements. Such composites may comprise intimate mixtures of materials of different type or may comprises ordered, arrays or layers or defined elements of different materials. “Polymers” in the context of the present invention include, but are not limited to conventional polymers and polymers produced by plasma deposition such as, polyolefins including low density polyethylene (LDPE), polypropylene (PP), high density polyethylene (HDPE), ultra-high molecular weight polyethylene (UHMWPE), blends of polyolefins with other polymers or rubbers; polyethers, such as polyoxymethylene (Acetal); polyamides, such as poly(hexamethylene adipamide) (Nylon 66); polyimides; polycarbonates; halogenated polymers, such as polyvinylidenefluoride (PVDF), polytetra-fluoroethylene (PTFE) (Teflon™), fluorinated ethylene-propylene copolymer (FEP), and polyvinyl chloride (PVC); aromatic polymers, such as polystyrene (PS); ketone polymers such as polyetheretherketone (PEEK); methacrylate polymers, such as polymethylmethacrylate (PMMA); polyesters, such as polyethylene terephthalate (PET); copolymers, such as ABS and ethylene propylene diene mixture (EPDM); degradable polymers such as polyesters including polycaprolactone, poly(lactic acid), poly(glycolic acid); and naturally-derived polymers such as silk and chitosan.

The term “co-deposition” as used herein refers to a deposition process which deposits at least two species on a surface simultaneously, which may involve varying over time the proportions of the two or more components to achieve graded layers of surface deposition. Most preferably the deposition of this graded layer is commenced with deposition of only the substrate material, noting that layers deposited prior to the deposition of carbon containing species become the effective substrate. This may result in a mixed or graded interface between materials.

By the term “mixed or graded interface” it is intended to denote a region in the material in which the relative proportions of two or more constituent components vary gradually according to a given profile. One method by which this mixed or graded interface is generated is by ion implantation. This achieves a transition from substrate material to deposited plasma polymer material. During the process any one of, or any combination of, the voltage, pulse length, frequency and duty cycle of the plasma immersion ion implantation (PIII) pulses applied to the substrate may vary in time thereby varying the extent to which the species arising from the plasma are implanted. Another example method by which a graded metal/plasma polymer interface can be achieved is co-deposition, where the power supplied to the magnetron or cathodic arc source of metal, or the composition of the gases supplied to the process chamber are varied so that the deposited and/or implanted material changes progressively from more metallic to more polymeric.

The term “plasma” or “gas plasma” is used generally to describe the state of ionised vapour. A plasma consists of charged ions, molecules or molecular fragments (positive or negative), negatively charged electrons, and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and vapour at low pressure is bled into the system. An electromagnetic field generated by a capacitive or inductive RF electrode is used to ionise the vapour. Free electrons in the vapour absorb energy from the electromagnetic field and ionise vapour molecules, in turn producing more electrons.

In conducting plasma treatment typically a plasma treatment apparatus (such as one incorporating a Helicon, parallel plate or hollow cathode plasma source or other inductively or capacitively coupled plasma source) is evacuated by attaching a vacuum nozzle to a vacuum pump. A suitable plasma forming vapour generated from a vapour, liquid or solid source is bled into the evacuated apparatus through a gas inlet until the desired vapour pressure in the chamber and differential across the chamber is obtained. An RF electromagnetic field is generated within the apparatus by applying current of the desired frequency to the electrodes from an RF generator. Ionisation of the vapour in the apparatus is induced by the electromagnetic field, and the resulting plasma modifies the metal, semiconductor, polymer, composite and/or ceramic substrate surface subjected to the treatment process. Preferred plasma forming gases that may be utilised are argon, nitrogen and organic precursor vapours as well as inorganic vapours consisting of the same or similar species as found in the substrate.

A plasma polymer surface can be generated through plasma ion implantation with carbon containing species, co-deposition under conditions in which substrate material is deposited with carbon containing species while gradually reducing substrate material proportion and increasing carbon containing species proportion and/or deposition of a plasma polymer surface layer with energetic ion bombardment. In this context the carbon containing species may comprise charged carbon atoms or other simple carbon containing molecules such as carbon dioxide, carbon monoxide, carbon tetrafluoride or optionally substituted branched or straight chain C₁ to C₁₂ alkane, alkene, alkyne or aryl compounds as well as compounds more conventionally thought of in polymer chemistry as monomer units for the generation of polymer compounds, such as n-hexane, allylamine, acetylene, ethylene, methane and ethanol. Additional suitable compounds may be drawn from the following non-exhaustive list: butane, propane, pentane, heptane, octane, cyclohexane, cycleoctane, dicyclopentadiene, cyclobutane, tetramethylaniline, methylcyclohexane and ethylcyclohexane, tricyclodecane, propene, allene, pentene, benzene, hexene, octene, cyclohexene, cycloheptene, butadiene, isobutylene, di-para-xylylene, propylene, methylcyclohexane, toluene, p-xylene, m-xylene, o-xylene, styrene, phenol, chlorphenol, chlorbenzene, fluorbenzene, bromphenol, ethylene glycol, diethlyene glycol, dimethyl ether, 2,4,6-trimethyl m-phenylenediamine, furan, thiophene, aniline, pyridine, benzylamine, pyrrole, propionitrole, acrylonitrile, pyrrolidine, butylamine, morpholine, tetrahydrofurane, dimethylformamide, dimethylsulfoxide, glycidyl methacrylate, acrylic acid, ethylene oxide, propylene oxide, ethanol, propanol, methanol, hexanol, acetone, formic acid, acetic acid, tetrafluormethane, fluorethylene, chloroform, tetrachlormethane, trichlormethane, trifluormethane, vinyliden chloride, vinyliden fluoride, hexamethyldisiloxane, triethylsiloxane, dioxane, perfluoro-octane, fluorocyclobutane, octafluorocyclobutane, vinyltriethoxysilane, octafluorotoluene, tetrafluoromethane, hexamethyldisiloxane, heptadecafluoro-1-decene, tetramethyldisilazane, decamethyl-cyclopentasiloxane, perfluoro(methylcyclohexane), 2-chloro-p-xylene.

Typical plasma treatment conditions (which are quoted here with reference to the power that may be required to treat a surface of 100 square centimetres, but which can be scaled according to the size of the system) may include power levels from about 1 watt to about 1000 watts, preferably between about 5 watts to about 500 watts, most preferably between about 30 watts to about 300 watts (an example of a suitable power is forward power of 100 watts and reverse power of 12 watts); frequency of about 1 kHz to 100 MHz, preferably about 15 kHz to about 50 MHz, more preferably from about 1 MHz to about 20 MHz (an example of a suitable frequency is about 13.5 MHz); axial plasma confining magnetic field strength of between about 0 G (that is, it is not essential for an axial magnetic field to be applied) to about 100 G, preferably between about 20 G to about 80 G, most preferably between about 40 G to about 60 G (an example of a suitable axial magnetic field strength is about 50 G); exposure times of about 5 seconds to 12 hours, preferably about 1 minute to 2 hours, more preferably between about 5 minutes and about 20 minutes (an example of a suitable exposure time is about 13 minutes); gas/vapour pressures of about 0.0001 to about 10 torr, preferably between about 0.0005 torr to about 0.1 torr, most preferably between about 0.001 torr and about 0.01 torr (an example of a suitable pressure is about 0.002 torr); and a gas flow rate of about 1 to about 2000 cm³/min.

The invention will now be further described with reference to the following non-limiting example.

### Example

### Materials and Methods

Selenium dioxide and polyvinyl alcohol (PVA, MW 9000-10000, 80% hydrolysed) were obtained from Sigma Aldrich (Castle Hill, NSW, Australia). Sodium thiosulphate was obtained from Science Supply Australia (Mitcham, VIC, Australia). ε-poly-L-lysine with average molecular weight of 3500-4500 Da was purchased from Carbosynth (Berkshire, UK). PBS tablets were purchased from Gibco (UK). All water used was purified by a Milli-Q water purification system (Merck Millipore, Massachusetts, USA) to a resistivity of ≥18.2 MΩ·cm.

### Synthesis of Se NPs and Se NP-ε-PL

In this work, a reduction approach was adopted for fabrication of Se nanoparticles. Selenium dioxide (SeO₂) was used as the selenite precursor and sodium thiosulfate (Na₂S₂O₃) was used as the reducing agent. PVA was weighed and dissolved into purified water (resistivity 18.2 MΩ cm at 25° C., Merck Millipore, Germany) to a concentration of 10 mg/mL. 10 mL of selenium dioxide with concentration of 5 mM was then added to 10 mL PVA solution, marked as solution A. Sodium thiosulfate was then weighed and dissolved into purified water to a concentration of 0.4 M. 10 mL sodium thio sulfate solution was added to solution A and stirred with a magnetic rotor. After reaction for 2 h, the reacted solution was immediately transferred to 1.7 mL centrifuge tubes, and 1 mL solution was added to each centrifuge tube. The tubes were centrifuged at the rate of 13000 rpm (or 15500 g) for 20 min. After centrifuging, the selenium nanoparticles remained on the bottom or wall of the centrifuge tubes. The reaction liquid was replaced with purified water, and the Se NPs were re-dispersed into purified water using a vortex mixer. The above procedures were repeated three times to rinse the Se NPs. On the third occasion, instead of purified water, Se NPs were re-dispersed into phosphate buffered saline (PBS) solution. The Se NPs were then sterilized by passage through a 0.22 μm filter.

In the preparation of Se NP-ε-PL, the sterilized Se NP solution was centrifuged and the PBS solution was removed. The Se NPs were re-dispersed into a 2 mg/mL sterilized ε-PL in water solution. After at least 8 h, the Se NPs with ε-PL solution was centrifuged, and the ε-PL solution was replaced with PBS solution and stored at 4° C. until use.

### Characterization of Se NPs and Se NP-ε-PL

The shape and size distribution of Se NPs and Se NP-ε-PL were observed and measured by transmission electron microscopy (TEM, TECNAI F20) using an accelerating voltage of 200 keV. The size distributions and zeta potentials of Se NPs and Se NP-ε-PL were measured using a Zetasizer (Malvern, ATA Scientific) at 25° C. Selenium was set as the material with a refractive index of 2.6 and an absorption of 0.5, and water was set as the dispersant with refractive index of 1.330, a viscosity of 0.8872 cP and a dielectric constant of 78.5 [39]. To measure the Se concentration of the Se NP suspensions and Se NP-ε-PL suspensions, the particles were dissolved in nitric acid, and inductively coupled plasma-optical emission spectrometry (ICP-OES, Varian 720-ES) was adopted to measure the Se concentration. A colorimetric method was adopted to measure the concentration of ε-PL [40]. For the colorimetric method, 80 μL trypan blue (Gibco, UK) solution was added to 1.92 mL sample solution. After 1h incubation in a water bath at 37° C. the sample solution was centrifuged at 13000 rpm (or 15500 g) for 5 minutes. The absorbance of the supernatant was tested using a UV-visible spectrophotometer (Varian 50Bio) with the wavelength range from 200 nm to 800 nm. There were peaks at the wavelength of 585 nm. Initially, a standard curve was produced using concentrations of ε-PL of from 0 μg/mL to 20 μg/mL and the concentration of ε-PL in the Se NP-ε-PL was determined by comparison with the standard curve.

### Cytotoxicity Tests

Two methods were adopted to test the cytotoxicity of Se NP-ε-PL, namely the Cell Counting Kit-8 (CCK-8) assay and Lactate Dehydrogenase (LDH) assay.

Cytotoxicity of Se NPs, Se NP-ε-PL and pure ε-PL was tested using human dermal fibroblast cells (HDF). HDF were cultured in Dulbecco's modified Eagle's medium (DMEM) with 10% foetal bovine serum (FBS), 100 U·mL⁻¹ penicillin and 100 μg·mL⁻¹ streptomycin at 37° C. in a humidified atmosphere of 5% CO₂. For the CCK-8 tests, filter sterilised Se NPs, Se NP-ε-PL and pure ε-PL in PBS suspensions were diluted in PBS to concentrations of 20, 50, 100, 200, 500 and 1000 μg/mL and 5% (v/v) of these diluted solutions were added to DMEM to give final concentrations of Se NPs of 1, 2.5, 5, 10, 25 and 50 μg/mL The control groups involved DMEM as the negative control and DMEM with 10% dimethyl sulfoxide (DMSO) as the positive control, according to ISO 10993-12 standard [41]. Cells were initially incubated in the 96-well plates at the density of approximately 4×10³ cells per 100 μL DMEM in each well and incubated for 24 h to allow attachment. The DMEM was then replaced by 100 μL DMEM with Se NPs, Se NP-ε-PL or pure ε-PL. After 24 h incubation, the medium was removed, and the cultures were washed once with PBS. 120 μL of DMEM with 10% CCK-8 solution was then added to each well and incubation was continued for 3 h. 100 μL medium from each well was then transferred to new 96-well plates and the absorbance of each well was tested using a microplate reader (M200 PRO, Tecan) at a wavelength of 450 nm. The cell viability (X) of each experimental group was calculated based on 5 samples according the following formula:

$$X = \frac{{OD}_{1}}{{OD}_{2}} \times 100 \% ,$$

where OD₁ represents the mean absorbance of experimental groups and the positive control group and OD₂ represents the mean absorbance of the negative control group.

LDH is released from cells into the medium when cell lysis occurs. The amount of LDH released to the medium was measured using the Cyto Tox 96® nonradioactive assay (Promega, Madison, Wis., USA) following the manufacturer's instructions. The HDF cells were cultured in a 96-well microplate for 24 h, before the old medium was replaced with DMEM with including either Se NPs, Se NP-ε-PL or pure ε-PL. Pure DMEM was used as negative control. The microplate was put back into the incubator for 6 h. For the maximum LDH release control, HDF cells were lysed using 1× lysis solution for 45 minutes before adding Cyto Tox 96 reagent. Cyto Tox 96 reagent was then added to the wells and the microplate was incubated for a further 30 minutes. The reaction was then stopped by adding stop solution. A microplate reader (M200 PRO, Tecan) was utilized to test absorbance at 490 nm. The cytotoxicity (Y) was calculated using the following formula:

$$Y = \frac{{OD}_{3}}{{OD}_{4}} \times 100 \% ,$$

OD3 represents the mean absorbance of experimental groups, and OD4 represents the mean absorbance of the maximum LDH release control group. All absorbance values are after subtraction of the culture medium background values.

### Antibacterial Tests

For testing the antibacterial activity of Se nanoparticles, two different methods—a bacterial growth inhibition test and a colony forming units (CFU) measuring assay were adopted.

### (a) Bacterial Growth Inhibition Test

The bacterial strains methicillin-sensitive Staphylococcus aureus (S. aureus) ATCC 29213, methicillin-resistant Staphylococcus aureus (MRSA) ATCC 43300, Enterococcus faecalis (E. faecalis) ATCC 29212, Escherichia coli (E. coli) ATCC 25922, Acinetobacter baumannii (A. baumannii) 2208 ATCC19606, Pseudomonas aeruginosa (P. aeruginosa) strain PAO1-LAC ATCC 47085, Klebsiella pneumoniae (K. pneumoniae) ATCC 13883 and multidrug-resistant K. pneumoniae (MDR), were obtained from the culture collection of the Oral Health Cooperative Research Centre, The Melbourne Dental School, University of Melbourne, Australia. Bacteria were cultured in Mueller Hinton Broth (MHB) at 37° C. 50 μL of suspensions with the desired concentrations of Se NPs, Se NP-ε-PL or pure ε-PL in PBS solution was added to each well of 96-well microplates, then 50 μL MHB with 2.5×10⁶ bacteria/mL were added into each well. The plate was put into an iEMS microplate reader (Pathtech Pty Ltd, Melbourne, Australia) at a temperature of 37° C. to monitor bacterial growth by measuring the absorbance at wavelength of 630 nm for 24 h. Background absorbance values due to the Se NP solutions were subtracted from the measured absorbance values.

### (b) Minimum Inhibitory Concentration (MIC) Calculation

The absorbance values from bacteria growth curves at the time when the stationary phase starts (tₛₚₛ) were taken and calculated as a percentage of the untreated control (Z=OD₅/OD₆×100%, where OD5 represents absorbance at tₛₚₛ of experimental groups and OD₆ represents the absorbance at tₛₚₛ of the negative control group; all absorbance values had the culture medium background values subtracted). Concentration-inhibition curves were plotted with the percentages of absorbance, and linear regression analysis was adopted to determine the MIC at which Z becomes zero.

### (c) CFU Measuring Assay

For the CFU measuring assay, 50 μL of different concentrations of Se NPs, Se NP-ε-PL or pure ε-PL in PBS solution was added into each well of 96-well microplates, then 50 μL MHB with 2.5×10⁶ cells/mL bacteria were added into each well. After incubating the microplate at 37° C. for 90 min, the bacterial solutions were diluted 10⁻¹, 10⁻², 10⁻³ and 10⁻⁴ times, then 10 μL of each dilution was transferred onto agar plates with MHB. The agar plates were incubated overnight, before the bacterial colony forming units were observed and measured.

### (d) Minimum Bactericidal Concentration (MBC) Calculation

Concentration-killing curves were plotted with CFUs/mL as a function of antibacterial agent concentration, and linear regression analysis was used to determine the lowest concentration (MBC) at which the CFU/mL becomes zero.

### Antibacterial Mechanism Tests

### (a) Adenosine Triphosphate (ATP) Tests

The bacterial strains methicillin-sensitive S. aureus ATCC 29213, E. faecalis ATCC 29212, E. coli ATCC 25922 and K. pneumoniae ATCC 13883 were cultured in MHB at 37° C. 50 μL of Se NPs, Se NP-ε-PL or pure ε-PL in PBS solution was added into each well of 96-well microplates. 50 μL MHB with 2.5×10⁶ cells/mL bacteria was then added into each well. After 1h incubation at 37° C., the 96-well microplates were transferred to room temperature for a further 30 min incubation period. 100 μL of BacTiter-Glo™ reagent (Promega, Australia) was added into each well, then mixed on an orbital shaker and incubated for 5 min. The luminescence was recorded using a microplate reader (PerkinElmer 1420 Multilabel Counter VICTOR3).

A standard curve was generated by adopting the following steps. 10-fold serial dilutions of ATP from 1 μM to 10 pM in 100 μL MHB were prepared. 100 μL of BacTiter-Glo™ reagent was added into each well, then mixed on an orbital shaker and incubated for 1 min. The luminescence was recorded using a microplate reader (PerkinElmer 1420 Multilabel Counter VICTOR3).

### (b) ROS Production Tests

The bacterial strains methicillin-sensitive S. aureus (MSSA) ATCC 29213, E. faecalis ATCC 29212, E. coli ATCC 25922 were cultured in MHB at 37° C. 50 μL of different concentration of Se NPs, Se NP-ε-PL or pure ε-PL in PBS solution was added into each well of a 96-well microplate. 50 μL MHB with 2.5×10⁶ cells/mL bacteria was then added into each well. After 90 min incubation at 37° C., CellROX® Orange Reagent was added into each well at a final concentration of 750 nM. The cells were incubated for a further 1 h. The fluorescence from the CellROX Orange Reagent was measured on FL-3 (red fluorescence channel) using the Cell Lab Quanta SC MPL flow cytometer (Beckman Coulter). Two independent experiments were done for this test, and two technical replicates were adopted for each independent experiment.

### (c) Membrane Potential Change Tests

Membrane potential change was measured using a BacLight Bacterial Membrane Potential Kit (Invitrogen). The bacterial strain methicillin-sensitive S. aureus (MSSA) ATCC 29213, E. faecalis ATCC 29212, E. coli ATCC 25922 were cultured in MHB at 37° C. 50 μL of different concentrations of Se NPs, Se NP-ε-PL or pure ε-PL in PBS solution was added into each well of 96-well microplates. 50 μL MHB with 2.5×106 cells/mL bacteria was then added into each well. 50 μL MHB with 2.5×106 cells/mL bacteria was added to 50 μL PBS to act as untreated control. Carbonyl cyanide 3-chlorophenylhydrazone (CCCP) was added to untreated control at a final concentration of 5 μM to act as fully depolarized control. 3,3′-Diethyloxacarbocyanine Iodide (DiOC2(3)) was added to all wells at a final concentration of 3 mM. The DiOC2(3) exhibits green fluorescence in all bacterial cells at low concentrations, but will be more concentrated in healthy bacteria cells where membrane potential is maintained, and fluorescence is shifted to red. After 1 h of incubation at 37° C., membrane potential was determined by a Cell Lab Quanta SC MPL flow cytometer (Beckman Coulter) as the ratio of cells that exhibited red fluorescence (FL-3) to those that displayed green fluorescence (FL-1). The untreated (polarized) and CCCP-treated (fully depolarized) controls were used to determine the percentage of depolarized cells. Two independent experiments were done for this test, and two technical replicates were adopted for each independent experiment.

### (d) Membrane Disruption Tests

The bacterial strains methicillin-sensitive S. aureus ATCC 29213, E. faecalis ATCC 29212, E. coli ATCC 25922, A. baumannii 2208 ATCC19606, P. aeruginosa strain PAO1-LAC ATCC 47085 and K. pneumoniae ATCC 13883 were cultured in MHB at 37° C. 50 μL of different concentrations of Se NPs, Se NP-ε-PL or pure ε-PL in PBS solution was added into each well of 96-well microplates. 50 μL MHB with 2.5×10⁶ cells/mL bacteria was then added into each well. 50 μL MHB with 2.5×10⁶ cells/mL bacteria was added to 50 μL PBS to act as untreated control. After 90 min incubation at 37° C., 0.1% of a green-fluorescent nucleic acid stain (SYTO 9, Thermo Fisher Scientific) and 0.1% of propidium iodide (PI) were added to each well and incubated again for 5 min. SYTO 9 can stain both live and dead Gram-positive and Gram-negative bacteria. PI is a red-florescent nuclear and chromosome counterstain but is not permeant to cells with intact plasma membranes. A Cell Lab Quanta SC MPL flow cytometer (Beckman Coulter) was adopted to measure the percent of PI-positive cells. Two independent tests were performed, and two parallel samples were used in each test for each variation.

### (d) HIM Images

The morphologies of bacterial strains methicillin-sensitive S. aureus (MSSA) ATCC 29213, E. faecalis ATCC 29212, E. coli ATCC 25922, A. baumannii 2208 ATCC19606 and K. pneumoniae ATCC 13883 after treatment with Se NPs, Se NP-ε-PL or pure ε-PL were observed using Helium Ion Microscopy (HIM, Zeiss, Germany). The samples were prepared in the following steps. First, 100 μL of 125 μg/mL Se NPs, Se NP-ε-PL or pure ε-PL in PBS solution was added to each well of 96-well microplates, 100 μL pure PBS was used as untreated control. Afterwards, 100 μL MHB with 1.25×10⁷/mL bacteria was added to each well. After 90 min incubation, 10 μL of both treated and untreated bacteria was dropped on clean silicon wafers, and they were then dried at 37° C. for 20 min. The dried samples were transferred to a 12-well plate, 2.5% glutaraldehyde was added to each well to fix the bacteria cells for 1 h, then gradient ethanol solution (30%, 50%, 60%, 70%, 80%, 90%, 95% and 100%) was used for dehydration. The prepared samples were dried in the fume hood overnight before use.

### (e) Bacterial Zeta Potential Measurements

The bacterial strains methicillin-sensitive S. aureus ATCC 29213, E. faecalis ATCC 29212, E. coli ATCC 25922, A. baumannii 2208 ATCC19606 and K. pneumoniae ATCC 13883 were inoculated from an agar plate into MHB for overnight culture. The obtained bacterial suspensions were centrifuged at 13500 rpm and 37° C. for 20 min, before being washed with sterilized water three times and then resuspended into PBS solution. 10 μL of the resuspended bacterial solution was diluted to 1 mL with PBS solution for zeta potential measurements. Each sample was measured three times.

### Resistance Tests

The resistance development of methicillin-sensitive S. aureus ATCC 29213 on Se NPs, Se NP-ε-PL and kanamycin and E. coli ATCC 25922 on Se NP-ε-PL and kanamycin were tested. First, one S. aureus or E. coli colony from an agar plate was inoculated into 20 mL MHB and cultured overnight at 37° C. The bacterial suspensions were diluted to 2.5×10⁶ bacteria/mL. 10 mL 2.5×10⁶ bacteria/mL with Se NPs or Se NP-ε-PL or kanamycin at MBC50 concentration (the concentration of antibacterial agent that kills 50% of bacteria) were cultured for 24 h (˜11 generations growth of S. aureus and ˜13 generations of E. coli, (number of generations)=Log₂((concentration of bacteria after 24 h culture)/(2.5×10⁶ bacteria/mL)). These bacterial suspensions were diluted again to 2.5×10⁶ bacteria/mL, and 10 mL 2.5×10⁶ bacteria/mL with Se NPs or Se NP-ε-PL or kanamycin at MBC50 concentration and were cultured for 24 h. These steps were repeated daily for in excess of 300 generations. After every 48 h, CFU assays were performed on these bacteria, and the MBCs were calculated. The change in MBCs over the generations was plotted.

### Statistical Analysis

Data in this work are expressed as means±standard deviation. Statistical analysis was performed by one-way analysis of variance (ANOVA) followed by Tukey's post hoc tests using SPSS 22.0 and p-values less than 0.05 were considered statistically significant.

### Results

Characterization of Se NPs with ε-PL

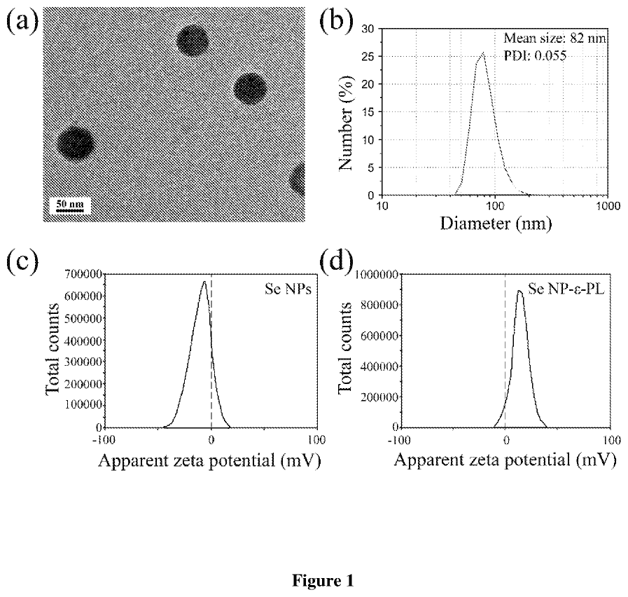

The TEM image of Se NP-ε-PL is shown in FIG. 1(a). FIG. 1 (b) shows the size distribution of Se NP-ε-PL. The mean size (diameter) of Se NP-ε-PL was 82 nm. The Se NPs adsorbed ε-PL on the surface through electrostatic adsorption, which resulted in an increase of the zeta potential of Se NPs from a negative value (−7.2±3.9 mV with PVA) to a positive value (+13.2±2.8 mV) (as shown in FIGS. 1 (c) and (d)).

Both the concentration of Se ions and of ε-PL of ten independently prepared Se NP-ε-PL solutions were measured, and the concentration ratios of Se NPs and ε-PL were between 1:0.85 and 1:1.09. In other words, 1 μg/mL Se NPs adsorbed 0.85-1.09 μg/mL ε-PL.

### Cytotoxicity Tests

Two methods—a Cell Counting Kit-8 (cck-8) assay and Lactate Dehydrogenase (LDH) assay, were adopted to test the cytotoxicity of Se NPs, Se NP-ε-PL and pure ε-PL. The Cell Counting Kit-8 test indicates cell viability, and the LDH test measures the degree of cell damage.

### Cell Proliferation Assay

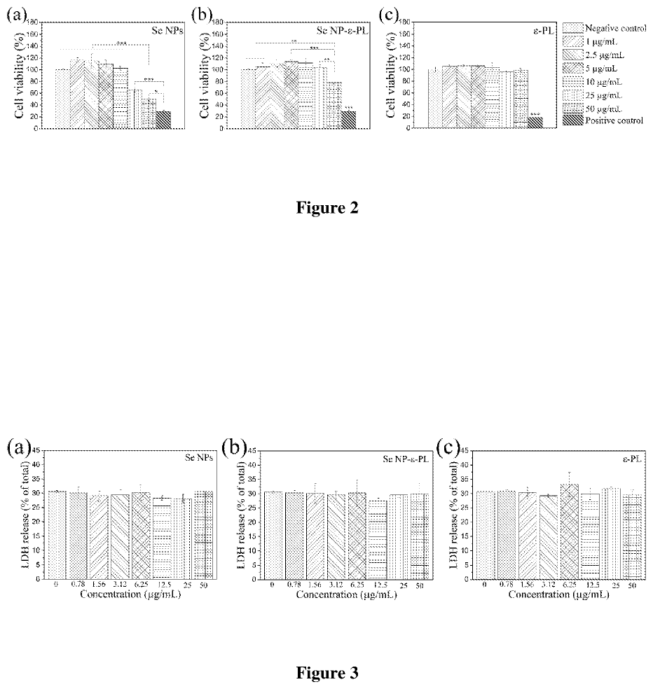

FIG. 2 shows cell viabilities of human dermal fibroblasts (HDF) after treatment with different concentrations of Se NPs, Se NP-ε-PL or pure ε-PL. ε-PL at all tested concentrations exhibited no significant toxicity to HDF cells. No obvious cytotoxicity was observed when adding lower than 10 μg/mL of Se NPs. Se NP-ε-PL showed no significant cytotoxicity up to and including a concentration of 25 μg/mL. For the Se NP-ε-PL, the weight concentration of Se is about half of the total concentration. Comparing the cytotoxicity results of Se NPs to those of Se NP-ε-PL, Se NPs and Se NP-ε-PL showed a similar toxicity to HDF cells when they were at the same Se concentration. Therefore, the cytotoxicity of Se NP-ε-PL was presumed mainly to derive from the Se NPs. Low doses of Se NPs or Se NP-ε-PL may in fact promote cell proliferation.

### LDH Test

The lactase dehydrogenase (LDH) assay was adopted to assess effects of the Se NPs, Se NP-ε-PL and pure ε-PL the with HDFs. LDH is an enzyme present within the mitochondria of living cells, so increased levels of the LDH in the culture medium imply rupture of the cell membranes. In this work, the level of LDH released from HDF cells into the medium was measured, and the results are shown in FIG. 3. Cells treated with Se NPs, Se NP-ε-PL or pure ε-PL for 6h showed no significant difference to the untreated control, indicating that Se NPs, Se NP-ε-PL and pure ε-PL, each at a concentration of up to 50 μg/mL will not cause obvious cell damage in a 6 h period.

### Antibacterial Activity Tests

### (a) Growth Inhibition Test Using Weight Concentration

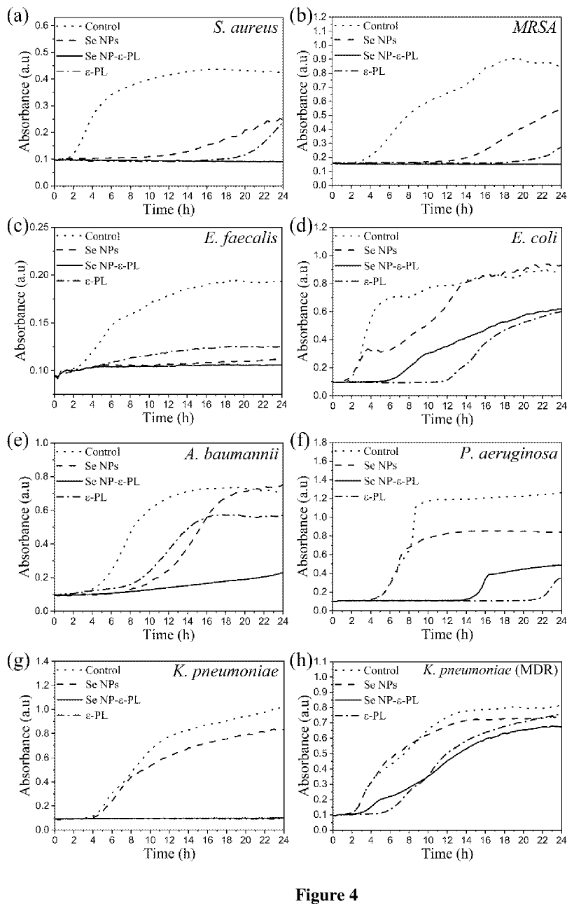

The growth inhibition effects of Se NPs, Se NP-ε-PL and pure ε-PL at concentrations from 1.56 to 100 μg/mL on various types of bacteria were tested. The growth curves with 12.5 μg/mL of Se NPs, Se NP-ε-PL and pure ε-PL clearly exhibited the different bacterial growth inhibition effects of these three materials (FIG. 4). The Se NP-ε-PL showed higher growth inhibition activity than pure Se NPs in all tested bacterial strains. For Gram-positive bacteria, Se NP-ε-PL showed better antibacterial efficacy than pure ε-PL. For Gram-negative bacteria, apart from slightly lower growth inhibition effects than pure ε-PL on E. coli and P. aeruginosa, Se NP-ε-PL showed similar or higher antibacterial efficacy than ε-PL on A. baumannii, K. pneumoniae and K. pneumoniae (MDR). The pure Se NPs showed no or very little growth inhibition of Gram-negative bacteria. At the same concentration, Se NP-ε-PL includes only half the amount of ε-PL as the pure ε-PL samples, but still showed very similar or even higher antibacterial activity on all tested types of bacteria.

From the growth curves of bacteria in MHB treated with a series of concentrations of Se NPs, Se NP-ε-PL or pure ε-PL, the Minimum Inhibitory Concentration (MIC) could be calculated and the results are shown in Table 1.

TABLE 1
The MIC values of Se NPs, Se NP-ε-PL and pure
ε-PL on different types of bacteria

| | MIC (μg/mL) | | |
|---|---|---|---|
| Strains | Se NPs | Se NP-ε-PL | ε-PL |
| S. aureus | 10.1 ± 6.6 | 6.0 ± 0.3 | 7.5 ± 1.0 |
| MRSA | 11.5 ± 4.8 | 8.6 ± 4.2 | 11.6 ± 2.0 |
| E. faecalis | 10.4 ± 5.8 | 9.4 ± 3.8 | 27.1 ± 2.2 |
| E. coli | 340 ± 200^{†} | 19.5 ± 9.2 | 13.2 ± 0.5 |
| A. baumannii | 229 ± 10^{†} | 13.8 ± 1.2 | 21.6 ± 7.0 |
| P. aeruginosa | 290 ± 66^{†} | 12.5 ± 0.5 | 9.5 ± 3.6 |
| K. pneumoniae | 255 ± 29^{†} | 12.3 ± 0.2 | 12.0 ± 0.6 |
| K. pneumoniae (MDR) | 363 ± 46^{†} | 26.2 ± 0.4 | 26.5 ± 0.5 |

* All data are expressed as mean ± standard deviation (s.d.) of the biological replicates.
^{†}As these sizes of Se NPs showed no total inhibition of growth or killing of bacteria within the tested concentrations, these MIC and MBC values, calculated through linear fitting, are only for comparison.

### (b) Colony Forming Unit Assay

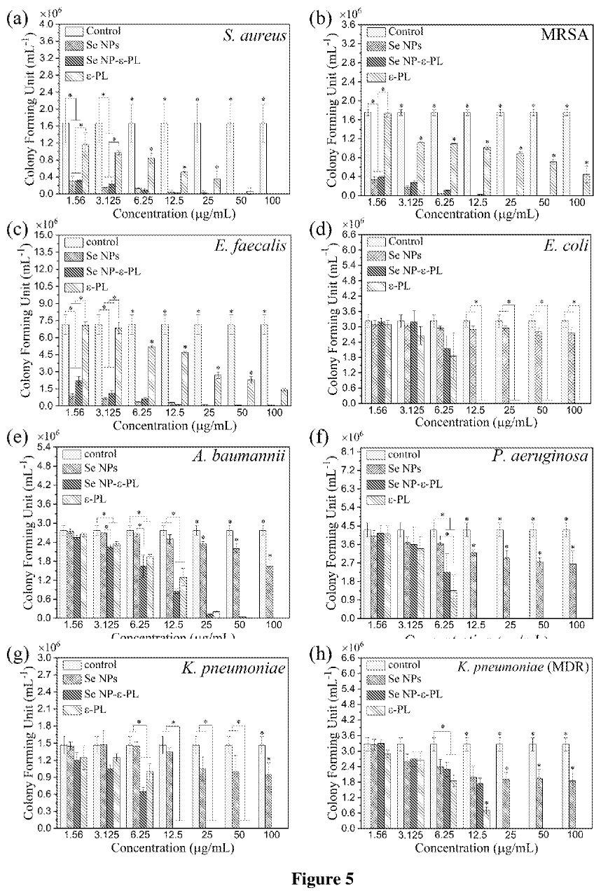

The bactericidal ability of Se NPs, Se NP-ε-PL and ε-PL on different types of bacteria were tested using a colony forming unit assay, the results of which are shown in FIG. 5. Both Se NPs and Se NP-ε-PL showed higher bactericidal effects on Gram-positive bacteria than ε-PL. For Gram-negative bacteria, Se NPs showed no or very little bactericidal effect, while both Se NP-ε-PL and ε-PL showed a high bactericidal efficacy on all tested Gram-negative bacteria (with no significant differences on most tested Gram-negative bacteria). The MBC values of Se NPs, Se NP-ε-PL and ε-PL on the tested bacterial strains were calculated and are shown in Table 2. The Se NP-ε-PL showed lower MBC values than both Se NPs and ε-PL on Gram-positive bacteria. For the Gram-negative bacteria, Se NP-ε-PL showed very similar MBC values to pure ε-PL, but much lower MBC values than those of Se NPs. As noted above, at the same concentration, the amount of ε-PL in the Se NP-ε-PL is only around half that in the pure ε-PL group. Despite this, they showed very similar MBC values on Gram-negative bacteria, demonstrating the efficacy of the Se NP-ε-PL relative to pure ε-PL. To investigate any cooperative actions achieved through the delivery of the ε-PL on the particles, a control experiment was performed to determine the MBC of E. faecalis when treated with equivalent amounts of Se NPs and free ε-PL (Se NPs+ε-PL) as used in the treatment with the Se NP-ε-PL. The MBC of the blended Se NPs+ε-PL against E. faecalis was found to be 42.1±3.7 μg/mL. This value is significantly lower than the MBC for either Se NPs (119±1 μg/mL) or ε-PL (134±10 μg/mL) alone. However, the MBC for the Se NP-ε-PL treatment was significantly lower again at 23.2±0.4 μg/mL. This result indicates that the delivery of the ε-PL on the Se NP surfaces enhances the antibacterial activity of the Se NP-ε-PL system.

TABLE 2
The MBC values of Se NPs, Se NP-ε-PL and pure
ε-PL on different types of bacteria

| | MBC (μg/mL) | | |
|---|---|---|---|
| Strains | Se NPs | Se NP-ε-PL | ε-PL |
| S. aureus | 35 ± 14 | 17.5 ± 7.8 | 50.6 ± 3.5 |
| MRSA | 22.2 ± 2.0 | 23.6 ± 1.0 | 149 ± 37 |
| E. faecalis | 119 ± 1 | 23.2 ± 0.4 | 134 ± 10 |
| E. coli | 1830 ± 830^{†} | 24.7 ± 0.6 | 24.3 ± 1.2 |
| A. baumannii | 440 ± 290^{†} | 63 ± 17 | 50.0 ± 0.2 |
| P. aeruginosa | 520 ± 290^{†} | 25.0 ± 0.2 | 25.0 ± 0.1 |
| K. pneumoniae | 660 ± 310^{†} | 12.6 ± 0.1 | 12.9 ± 0.4 |
| K. pneumoniae (MDR) | 1140 ± 160^{†} | 25.0 ± 1.1 | 24.7 ± 0.5 |

* All data are expressed as mean ± standard deviation (s.d.) of the biological replicates.
^{†}As these sizes of Se NPs showed no total inhibition of growth or killing of bacteria within the tested concentrations, these MIC and MBC values, calculated through linear fitting, are only for comparison.

### Investigation of Antibacterial Mechanism of Se NPs

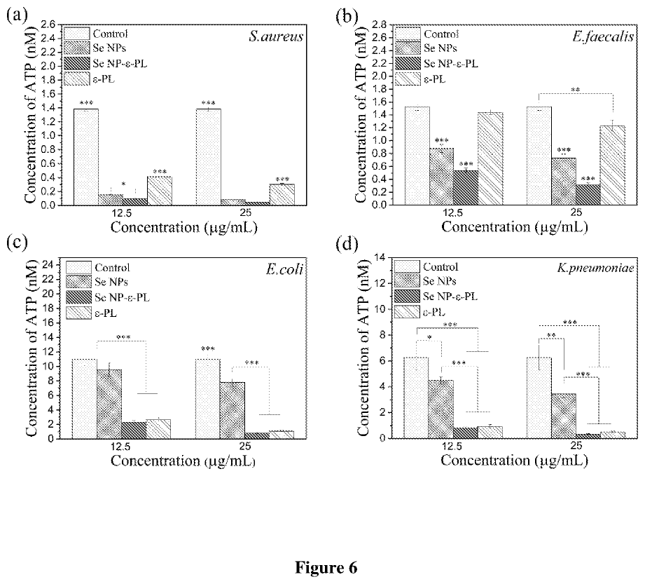

Adenosine triphosphate (ATP) is the intracellular energy molecule used by all living organisms. It plays a vital role in respiration and metabolism as it is the most important energy supplier for many enzymatic reactions. Its critical role makes it extremely important to cells [42]. We therefore investigated the effects of Se NPs, Se NP-ε-PL and pure ε-PL on ATP levels in S. aureus, E. faecalis E. coli and K. pneumoniae with bacteria in pure MHB as a control (FIG. 6). For the Gram-positive bacteria S. aureus and E. faecalis, Se NPs showed better ATP depletion activity than ε-PL. On the contrary, ε-PL exhibited a higher ATP depletion effect than Se NPs on the Gram-negative bacteria E. coli and K. pneumoniae. However, the Se NP-ε-PL showed the highest ATP depletion ability among the three materials tested. The depletion of cellular ATP within bacterial cells is a characteristic of energy-uncoupling effects, suggesting a potential mechanism by which Se NPs interfere with cellular metabolism [43].

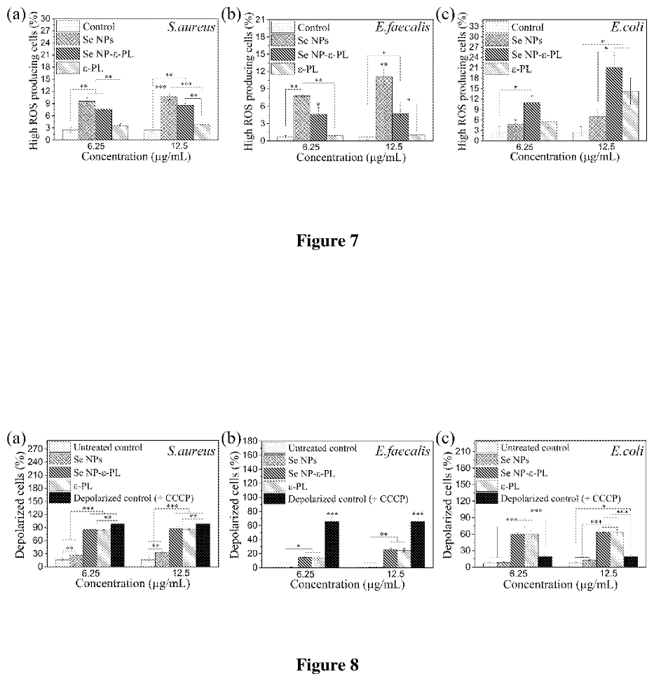

The oxidative stress induced by high ROS production in response to nanoparticles is considered to be an important mechanism of bacterial death [44]. Therefore, we next investigated the effect of Se NPs, Se NP-ε-PL and pure ε-PL on ROS production of S. aureus, E. faecalis and E. coli cells (FIG. 7), with bacteria in pure MHB as a control. Se NPs significantly promoted ROS production in the Gram-positive bacteria S. aureus and E. faecalis. Higher concentrations of Se NPs led to more high ROS production cells. ε-PL showed a slight promotion of ROS production in S. aureus cells, and almost no promoting effects on ROS production in E. faecalis cells. Se NP-ε-PL showed a moderate effect between those of Se NPs and ε-PL on S. aureus and E. faecalis cells in terms of ROS production. For the Gram-negative bacteria E. coli, Se NP-ε-PL showed the highest efficacy on promotion of ROS production in comparison to both pure Se NPs and pure ε-PL.

Depolarisation of cell membranes is a well-established mechanism of action of antimicrobial agents. Therefore, we next investigated the effects of Se NPs on the polarity of the cell membrane in bacterial cells (FIG. 8), with bacteria in pure MHB as the untreated control and bacteria treated with CCCP as the depolarized control. Se NPs only showed a slight effect in depolarizing S. aureus cells, and no effect on E. facecalis and E. coli cells. Se NP-ε-PL and pure ε-PL showed very similar effects, both depolarized cell membranes of the three types of bacteria tested—S. aureus, E. faecalis and E. coli. Interestingly, CCCP is a standard depolarising agent, yet shows minimal activity towards E. coli in contrast to the Se NP-ε-PL which showed strong depolarisation activity.

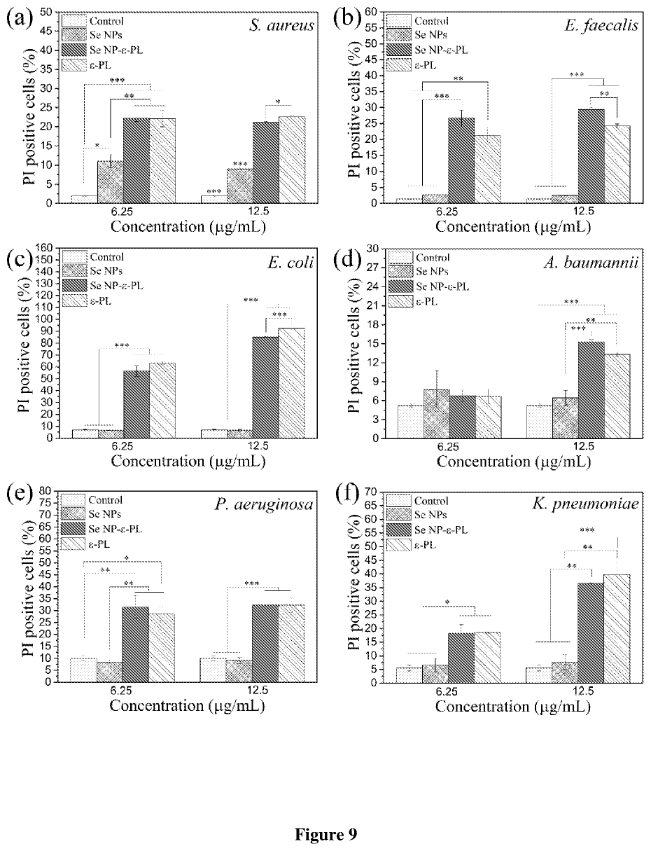

The percentage of propidium iodide (PI) positive cells results are shown in FIG. 9. PI positive cells represent membrane disrupted cells as PI can only penetrate damaged cell membranes [45]. Se NPs showed a slight effect only on S. aureus cell membrane disruption. However, Se NP-ε-PL and pure ε-PL showed membrane disruption effects on all tested bacterial strains.

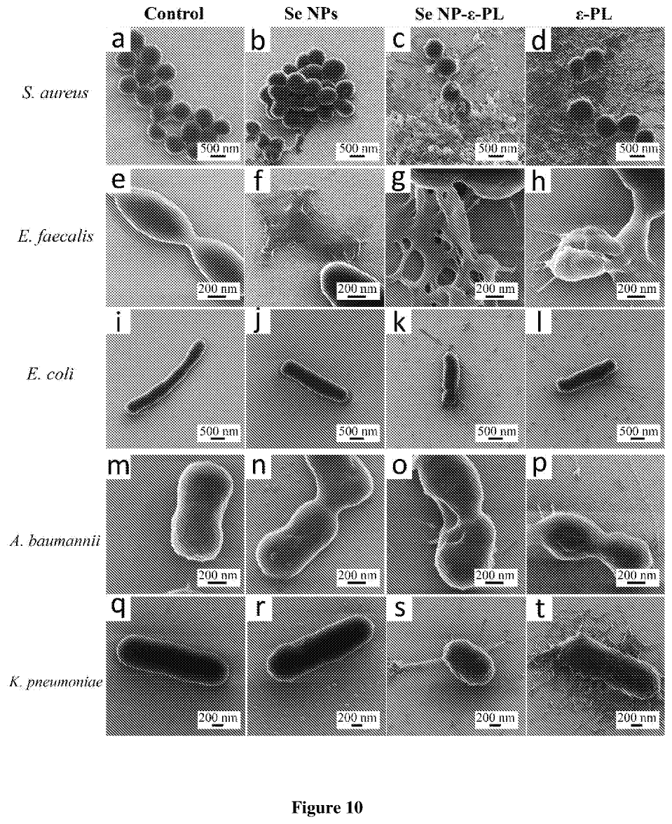

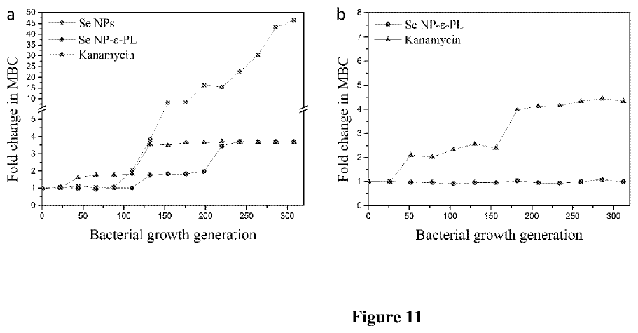

Helium ion microscopy (HIM) was adopted to observe S. aureus, E. faecalis, E. coli, A. baumannii and K. pneumoniae cells after treatment with Se NPs, Se NP-ε-PL or pure ε-PL, with bacteria in pure MHB as a control (FIG. 10). The untreated S. aureus cells were spherical with smooth surfaces. For the S. aureus cells treated with Se NPs, many cells showed changes in their shape or surface morphology. Most Se NP-ε-PL treated S. aureus cells were killed, while for the remaining cells, Se NP-ε-PL appeared to be attached to the bacteria (shown by pink arrows) and the bacterial surfaces were rough, indicating that these cells were also damaged. Normal E. faecalis cells are fusiform in shape. With treatment by Se NPs, some of the E. faecalis cells were killed and became flat. Most cells were dead after treatment with Se NP-ε-PL, and the remaining cells had NPs attached. For the ε-PL treated cells, some were dead and distorted. For Gram-negative cells, E. coli seem able to repel the Se NPs, and it appeared that the Se NPs did not attach to the E. coli cells. However, some Se NPs appeared to attach to A. baumannii and K. pneumoniae cells. The Se NPs did not cause visible damage to these bacterial cells. The membranes of E. coli, A. baumannii and K. pneumoniae cells were disrupted after treatment with ε-PL, with a resulting change in cell shapes. The Se NPs combined with ε-PL attached to E. coli cells and induced clearly visible damage to these cells. Compared to pure Se NPs, more Se NP-ε-PL appeared to attach to A. baumannii and K. pneumoniae cells and induced a higher degree of shape change than pure Se NPs.

From the zeta potential measurements results (Table 3), it can be seen that S. aureus, E. faecalis and A. baumannii have zeta potentials around −10 mV, which is around half of the values for E. coli and K. pnemoniae. As the Se NPs produced in this work are also negatively charged, the Se NPs can still affect S. aureus and E. faecalis cells (as shown in FIGS. 10 (b) and (f), respectively) and appeared to attach to the surface of A. baumannii cells (as shown in FIG. 10 (n)). However, they did not appear to get close to E. coli and K. pneumoniae cells (as shown in FIGS. 10 (j) and (r)). As a result of the influence of ε-PL, Se NP-ε-PL has a positively charged surface, which enables it to approach and attach or penetrate bacterial cells (as shown in FIGS. 10 (c), (g), (k), (o) and (s)).

TABLE 3
Zeta potentials of different types of bacteria

| Bacteria | S. | E. | E. | A. | K. |
|---|---|---|---|---|---|
| type | aureus | faecalis | coli | baumannii | pnemoniae |
| Mean zeta | −8.9 | −10.17 | −19.20 | −9.05 | −21.37 |
| potential | (±2.6) | (±0.65) | (±0.37) | (±0.22) | (±0.83) |
| (mV) | | | | | |

### Resistance Development Test

The MBC of kanamycin for S. aureus began to rise due to resistance to kanamycin after only 44 generations (FIG. 11 (a)), S. aureus started to develop resistance to Se NPs from 110 generations, and to Se NP-ε-PL from 132 generations (FIG. 11 (a)). E. coli started to develop resistance to kanamycin from 52 generations, but did not develop resistance to Se NP-ε-PL over the entire 312 generations tested (FIG. 11 (b)).

### Discussion

### Cytotoxicity of Se NPs

Based on the cytotoxicity test results, the toxicity of Se NP-ε-PL on human dermal fibroblasts at higher doses was mainly from Se NPs. Se NP-ε-PL exhibited very similar cytotoxicity to pure Se NPs when they were at the same Se concentration. Selenium is a trace element in the human body. Unlike nanoparticles made of non-nutritive elements such as Ag and Au nanoparticles, which exhibit higher cytotoxicity as concentration is increased [46], Se nanoparticles appear to even promote cell viability at relatively low concentrations.

### Antibacterial Mechanism of Se NP-ε-PL

ε-PL is a simple natural antimicrobial peptide (AMP) with 25-30 L-lysine residues [32]. The antibacterial mechanism of AMPs largely derives from the positive charge [47], which can disrupt the negatively charged membrane of microorganisms. However, this takes place only when there is a threshold concentration of AMPs binding to the cell membrane [29]. The most widely accepted models for the mechanism of AMPs disrupting bacterial membrane are the barrel-stave model and the carpet model. In the barrel-stave model, the AMPs firstly attach to the bacterial membrane, before the attached peptides aggregate together and penetrate the membrane bilayer. The hydrophobic parts of the AMPs combine with the lipid cores, leaving the hydrophilic parts of the AMPs to form a hole in the bacterial membrane. In the carpet model, the AMPs attach parallel to the surface of the bacterial membrane, then form a layer like a carpet to disrupt the membrane. In the present invention, it is understood without wishing to be bound by theory, that relative to free Se NPs and free AMPs, antimicrobial activity of Se NP-ε-PLs will be improved and the concentration of free AMPs will be decreased, to thereby reduce cytotoxicity on human cells.

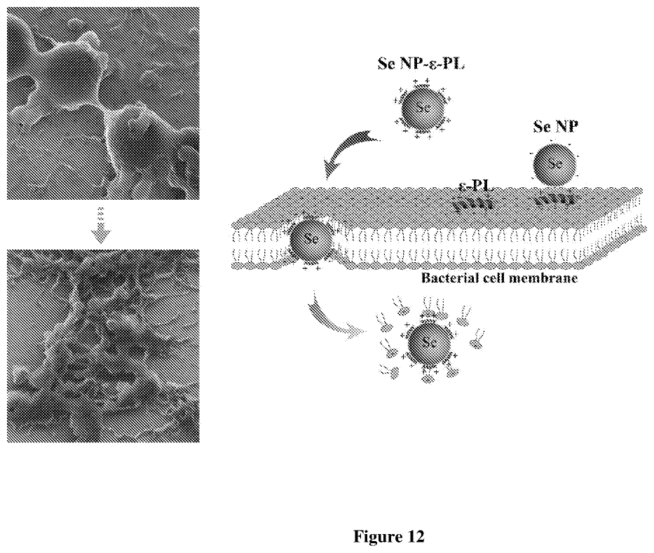

The Se NP-ε-PL displayed an effective broad-spectrum antimicrobial activity with delayed or no development of antimicrobial resistance and the antimicrobial mechanism is hypothesized as shown in FIG. 12. Again, without wishing to be bound by theory, it is understood that many ε-PL molecules were adsorbed on the surfaces of Se NPs by electrostatic attraction. These ε-PL molecules changed the zeta potential of Se NPs from a negative value (−7.2±3.9 mV with PVA capping) to a positive value (+13.2±2.8 mV). Generally, the surface potential of bacterial membranes is negative [48], although some bacteria exhibit lower negative membrane surface potential than others. For bacteria with relatively lower negatively charged membranes, Se NPs could still interact with the cell membrane (such as in S. aureus and E. faecalis, as shown in FIGS. 11 (b) and (f), respectively) or may attach to the bacterial surface (such as in A. baumannii, as shown in FIG. 11 (n)), but bacteria with relatively higher negatively charged membranes can repel the Se NPs away (such as in E. coli, as shown in FIG. 11 (j)). Since A. baumannii is a type of Gram-negative bacteria, it has a double membrane system [49]. So, even with Se NPs attached to the outer membrane of these bacteria, they could still be protected from the damage of Se NPs through the double membrane system. As K. pneumoniae has a capsule outside to protect itself [50], it may also be protected from Se NPs attached to the capsule surface (as shown in FIG. 11 (r)). However, the positively charged Se NP-ε-PL penetrated cells or attached on the surface of different types of bacteria much more readily (as shown in FIG. 11 (c), (g), (k), (o) and (s)). In Se NP-ε-PL, Se NPs also resulted in localisation of ε-PL molecules together to enhance their disruptive ability towards bacteria membranes.

The Se NP-ε-PL particles showed strong antibacterial properties at significantly lower AMP concentrations. This is likely due to the local high concentration of AMP present at the particle interface. Membrane disruption models require a threshold concentration of AMP to be present before membrane disruption can occur. By adsorbing the AMP molecules to the particle surface, regions of high AMP concentration are created, and these regions of local high density are likely able to disrupt the bacterial membrane, even when the bulk concentration of AMP is below the threshold point. This also explains why the Se NP-ε-PL particles showed greater antibacterial properties compared to treatment with Se NPs and free ε-PL. This mechanism is similar to the structurally nanoengineered antimicrobial peptide polymers (SNAPPs) which were developed recently [31]. Lam et al. adopted a dendrimer as a core to synthesize 16 or 32 peptides arms, which showed very high antibacterial efficacy on many types of bacteria, including Gram-negative bacteria.

The development of bacterial resistance to nanoparticles is generally considered less likely than to conventional antibiotics because the nanoparticles can kill bacteria through multiple mechanisms of action. Therefore, the multiple mechanisms of action used by the Se NP-ε-PL to kill bacteria are expected to limit the development of antimicrobial resistance in comparison to many antibiotics that exert their antibacterial influence through a single mechanism. To illustrate this, kanamycin was selected as the antibiotic control in this work. Kanamycin exerts its antibacterial properties through a single mechanism, interfering with protein synthesis by binding to the bacterial ribosome.

### CONCLUSION

In this work, Se NP-ε-PL were fabricated, and their cytotoxicity and antibacterial activity were assessed. The effects of Se NP-ε-PL on human dermal fibroblasts were found to mainly result from the Se in the NPs. Se NP-ε-PL exhibited antibacterial activities on all eight tested bacterial strains, including drug-resistant bacterial types. Considering antibacterial efficacy on both Gram-positive and Gram-negative bacteria, Se NP-ε-PL was found to be better than both Se NPs only and ε-PL only. It was shown that bacteria are much less likely to develop resistance to Se NP-ε-PL in comparison to the traditional antibiotic kanamycin. The efficient and broad-spectrum antibacterial activity and reduced likelihood of resistance render Se NP-ε-PL suitable candidates as new generation antimicrobial agents.

### REFERENCES

1. Brown, E. D. and G. D. Wright, Antibacterial drug discovery in the resistance era. Nature, 2016. 529(7586): p. 336-343.
2. Theuretzbacher, U., Global antibacterial resistance: The never-ending story. Journal of global antimicrobial resistance, 2013. 1(2): p. 63-69.
3. World-Health-Organisation, (Ed: W. H. Organisation), Geneva 2019
4. Liu, Y., et al., Antibacterial activities of zinc oxide nanoparticles against Escherichia coli O157: H7. Journal of Applied Microbiology, 2009. 107(4): p. 1193-1201.
5. Chamundeeswari, M., et al., Preparation, characterization and evaluation of a biopolymeric gold nanocomposite with antimicrobial activity. Biotechnology and applied biochemistry, 2010. 55(1): p. 29-35.
6. Huang, W. C., P. J. Tsai, and Y. C. Chen, Multifunctional Fe3O4@ Au Nanoeggs as Photothermal Agents for Selective Killing of Nosocomial and Antibiotic-Resistant Bacteria. Small, 2009. 5(1): p. 51-56.
7. Tiwari, P. M., et al., Functionalized gold nanoparticles and their biomedical applications. Nanomaterials, 2011. 1(1): p. 31-63.
8. Adams, C. P., et al., Size-dependent antimicrobial effects of novel palladium nanoparticles. PLoS One, 2014. 9(1): p. e85981.
9. Allahverdiyev, A. M., et al., Antimicrobial effects of TiO2 and Ag2O nanoparticles against drug-resistant bacteria and leishmania parasites. Future microbiology, 2011. 6(8): p. 933-940.
10. Jin, T. and Y. He, Antibacterial activities of magnesium oxide (MgO) nanoparticles against foodborne pathogens. Journal of Nanoparticle Research, 2011. 13(12): p. 6877-6885.
11. Jeong, M. S., et al., Characterization of antibacterial nanoparticles from the scallop, Ptinopecten yessoensis. Bioscience, biotechnology, and biochemistry, 2007. 71(9): p. 2242-2247.
12. Jones, N., et al., Antibacterial activity of ZnO nanoparticle suspensions on a broad spectrum of microorganisms. FEMS microbiology letters, 2008. 279(1): p. 71-76.
13. Kühn, K. P., et al., Disinfection of surfaces by photocatalytic oxidation with titanium dioxide and UVA light. Chemosphere, 2003. 53(1): p. 71-77.
14. Buckley, J. J., et al., Silver carbonate nanoparticles stabilised over alumina nanoneedles exhibiting potent antibacterial properties. Chemical Communications, 2008(34): p. 4013-4015.
15. Bondarenko, O., et al., Toxicity of Ag, CuO and ZnO nanoparticles to selected environmentally relevant test organisms and mammalian cells in vitro: a critical review. Archives of toxicology, 2013. 87(7): p. 1181-1200.
16. Tran, P. A. and T. J. Webster, Selenium nanoparticles inhibit Staphylococcus aureus growth. Int J Nanomedicine, 2011. 6: p. 1553-1558.
17. Cousins, B., et al., Effects of a nanoparticulate silica substrate on cell attachment of Candida albicans. Journal of applied microbiology, 2007. 102(3): p. 757-765.
18. Dhapte, V., et al., Versatile SiO 2 Nanoparticles@ Polymer Composites with Pragmatic Properties. ISRN Inorganic Chemistry, 2014. 2014.
19. Lyon, D. Y., et al., Bacterial cell association and antimicrobial activity of a C60 water suspension. Environmental Toxicology and Chemistry, 2005. 24(11): p. 2757-2762.
20. Qi, L., et al., Preparation and antibacterial activity of chitosan nanoparticles. Carbohydrate research, 2004. 339(16): p. 2693-2700.
21. Li, Q., et al., Antimicrobial nanomaterials for water disinfection and microbial control: potential applications and implications. Water research, 2008. 42(18): p. 4591-4602.
22. Mühling, M., et al., An investigation into the effects of silver nanoparticles on antibiotic resistance of naturally occurring bacteria in an estuarine sediment. Marine environmental research, 2009. 68(5): p. 278-283.
23. Kittler, S., et al., Toxicity of silver nanoparticles increases during storage because of slow dissolution under release of silver ions. Chem. Mater, 2010. 22(16): p. 4548-4554.
24. Kim, S., et al., Oxidative stress-dependent toxicity of silver nanoparticles in human hepatoma cells. Toxicology in vitro, 2009. 23(6): p. 1076-1084.
25. Underwood, E., Trace elements in human and animal nutrition. 2012: Elsevier.
26. Zasloff, M., Magainins, a class of antimicrobial peptides from Xenopus skin: isolation, characterization of two active forms, and partial cDNA sequence of a precursor. Proceedings of the National Academy of Sciences, 1987. 84(15): p. 5449-5453.
27. Bulet, P., R. Stocklin, and L. Menin, Anti-microbial peptides: from invertebrates to vertebrates. Immunological reviews, 2004. 198(1): p. 169-184.
28. Duclohier, H., G. Molle, and G. Spach, Antimicrobial peptide magainin I from Xenopus skin forms anion-permeable channels in planar lipid bilayers. Biophysical Journal, 1989. 56(5): p. 1017-1021.
29. Huang, H. W., Molecular mechanism of antimicrobial peptides: the origin of cooperativity. Biochimica et Biophysica Acta (BBA)-Biomembranes, 2006. 1758(9): p. 1292-1302.
30. Hancock, R. E. and H.-G. Sahl, Antimicrobial and host-defense peptides as new anti- infective therapeutic strategies. Nature biotechnology, 2006. 24(12): p. 1551-1557.
31. Lam, S. J., et al., Combating multidrug-resistant Gram-negative bacteria with structurally nanoengineered antimicrobial peptide polymers. Nature Microbiology, 2016. 1: p. 16162.
32. Yu, H., Y. Huang, and Q. Huang, Synthesis and characterization of novel antimicrobial emulsifiers from ε-polylysine. Journal of agricultural and food chemistry, 2009. 58(2): p. 1290-1295.
33. Shih, L., M.-H. Shen, and Y.-T. Van, Microbial synthesis of poly (ε-lysine) and its various applications. Bioresource Technology, 2006. 97(9): p. 1148-1159.
34. Geornaras, I., et al., Antimicrobial Activity of ε-Polylysine against Escherichia coli O157: H7, Salmonella Typhimurium, and Listeria monocytogenes in Various Food Extracts. Journal of Food Science, 2007. 72(8).
35. Najjar, M. B., D. Kashtanov, and M. Chikindas, ε-Poly-l-lysine and nisin A act synergistically against Gram-positive food-borne pathogens Bacillus cereus and Listeria monocytogenes. Letters in applied microbiology, 2007. 45(1): p. 13-18.
36. Hiraki, J., et al., Use of ADME studies to confirm the safety of ε-polylysine as a preservative in food. Regulatory Toxicology and Pharmacology, 2003. 37(2): p. 328-340.
37. Hyldgaard, M., et al., Binary combination of epsilon-poly-l-lysine and isoeugenol affect progression of spoilage microbiota in fresh turkey meat, and delay onset of spoilage in Pseudomonas putida challenged meat. International journal of food microbiology, 2015. 215: p. 131-142.
38. Shima, S., et al., Antimicrobial action of ε-poly-L-lysine. The Journal of antibiotics, 1984. 37(11): p. 1449-1455.
39. Hinterwirth, H., et al., Comparative method evaluation for size and size-distribution analysis of gold nanoparticles. Journal of separation science, 2013. 36(17): p. 2952-2961.
40. Shen, W.-C., D. Yang, and H. J.-P. Ryser, Colorimetric determination of microgram quantities of polylysine by trypan blue precipitation. Analytical biochemistry, 1984. 142(2): p. 521-524.
41. ISO, E., 10993-12: 2008—Biological evaluation of medical devices—Part 12: Sample preparation and reference materials (ISO 10993-12: 2007). German version: DIN EN ISO, 2008: p. 10993-12.
42. Mempin, R., et al., Release of extracellular ATP by bacteria during growth. BMC microbiology, 2013. 13(1): p. 301.
43. Lok, C.-N., et al., Silver nanoparticles: partial oxidation and antibacterial activities. JBIC Journal of Biological Inorganic Chemistry, 2007. 12(4): p. 527-534.
44. Li, Y., et al., Mechanism of photogenerated reactive oxygen species and correlation with the antibacterial properties of engineered metal-oxide nanoparticles. ACS nano, 2012. 6(6): p. 5164-5173.
45. O'Brien-Simpson, N. M., et al., A rapid and quantitative flow cytometry method for the analysis of membrane disruptive antimicrobial activity. PloS one, 2016. 11(3): p. e0151694.
46. Yen, H. J., S.h. Hsu, and C. L. Tsai, Cytotoxicity and immunological response of gold and silver nanoparticles of different sizes. Small, 2009. 5(13): p. 1553-1561.
47. Kohanski, M. A., D. J. Dwyer, and J. J. Collins, How antibiotics kill bacteria: from targets to networks. Nature Reviews Microbiology, 2010. 8(6): p. 423-435.
48. Dickson, J. S. and M. Koohmaraie, Cell surface charge characteristics and their relationship to bacterial attachment to meat surfaces. Applied and environmental microbiology, 1989. 55(4): p. 832-836.
49. Nikaido, H., Multidrug efflux pumps of gram-negative bacteria. Journal of bacteriology, 1996. 178(20): p. 5853.
50. SPRINGER, E. L. and I. Roth, The ultrastructure of the capsules of Diplococcus pneumoniae and Klebsiella pneumoniae stained with ruthenium red. Microbiology, 1973. 74(1): p. 21-31.
51 Vandati, M.; Moghadam, T. T. Synthesis and Characterization of Selenium Nanoparticles-Lysozyme Nanohybrid System with Synergistic Antibacterial Properties. Sci. Rep. 2020, 10 (1), 1-10.

1. An antimicrobial agent comprising a selenium nanoparticle (SeNP) core and one or more superficially located antimicrobial peptide/s (AMP).

2. The agent of claim 1 that is substantially spherical.

3. The agent of claim 2, having mean particle diameter of from about 10 nm to about 400 nm.

4. The agent of claim 1, wherein the one or more AMP each independently comprises an average of from about 5 to about 120 amino acids.

5. The agent of claim 1, wherein the one or more AMP each independently comprises an excess of positively charged amino acids compared to negatively charged amino acids.

6. The agent of claim 1, wherein the one or more AMP each independently comprises at least one peptide selected from polylysine, polyarginine, aurein, ovispirin, melittin, magainin, cecropin, andropin, moricin, ceratotoxin, melittin, magainin, dermaseptin, bombinin, brevinin, esculentins, buforin, cathelicidin, abaecin, apidaecin, prophenin and indolicidin.

7. The agent of claim 1, wherein the one or more AMP comprises ε-poly-_{L}-lysine (ε-PL).

8. The agent of claim 1, wherein the one or more AMP is electrostatically adsorbed to the SeNP core.

9. The agent of claim 1, wherein the one or more AMP is hydrogen bonded to the SeNP core or to a linker or stabiliser agent attached to the core.

10. The agent of claim 1, wherein the one or more AMP is covalently bound to a linker or stabiliser agent attached to the core.

11. An antimicrobial composition, comprising the antimicrobial agent of claim 1 and one or more carrier, diluent or vehicle.

12. A method of synthesis of an antimicrobial agent that comprises a selenium nanoparticle (SeNP) core and one or more superficially located antimicrobial peptide/s (AMP), said method comprising dispersing Se NPs into a solution of the one of more AMP and recovering the antimicrobial agent produced.

13. A method of killing or retarding growth of a microorganism, the method comprising exposing a microorganism or its locus to the antimicrobial agent of claim 1.

14. The method of claim 13 wherein the microorganism is Gram-positive bacteria, Gram-negative bacteria, fungi, enveloped virus, algae, protist, helminth, protozoa, archaea, rotifer or planaria.

15. The method of claim 13 wherein the microorganism is Gram-positive bacteria or Gram-negative bacteria.

16-18. (canceled)

19. An article that incorporates the antimicrobial agent of claim 1 on a surface thereof that may be exposed to a microorganism, or that enables agent release/exposure to a microorganism, wherein said article comprises a cleaning or disinfecting formulation, coating composition, textile, clothing, protective clothing, mask, furniture, a building fitting or fixture, a food preparation surface, utensil or apparatus, a food or beverage packaging, processing or storage container, food wrap, a wound dressing or a medical, surgical, veterinary or dental implant, tool or instrument.

20. The agent of claim 2, having mean particle diameter of from about 50 nm to about 200 nm.

21. The agent of claim 2, having mean particle diameter of from about 70 nm to about 120 nm.

22. The agent of claim 1, wherein the one or more AMP each independently comprises an average of from about 10 to about 100 amino acids.

23. The agent of claim 1, wherein the one or more AMP each independently comprises an average of from about 20 to about 60 amino acids.
